(19) United States
(12) Patent Application Publication
(10) Pub. No.: US 2021/0263028 A1
(43) Pub. Date: Aug. 26, 2021

(54) ASSAY

(71) Applicant: LumiraDx UK Ltd., London (GB)

(72) Inventors: Phill Lowe, Stirling (GB); Steven Alexander Keatch, Stirling (GB); Brian McGuigan, Stirling (GB); Lois Bello, Stirling (GB); Chris Slevin, Stirling (GB); David Lang, Stirling (GB); John Dilieen, Stirling (GB)

(21) Appl. No.: 17/275,922

(22) PCT Filed: Sep. 13, 2019
(86) PCT No.: PCT/GB2019/052573

§ 371 Date: Mar. 12, 2021

(30) Foreign Application Priority Data
Sep. 19, 2018 (GB) 1815278.5
Aug. 9, 2019 (GB) 1911397.6

(51) Int. Cl. G01N 33/543; B01L 3/00; B01L 7/00

(52) U.S. Cl. CPC G01N 33/54386; B01L 3/52; G01N 33/54393; B01L 7/52; B01L 3/502715

(57) ABSTRACT

The present invention relates to methods for detecting an analyte present in a fluid sample using a microfluidic device comprising a detection zone characterized by an optically transmissible portion and reagent(s) associated with a porous matrix, wherein the analyte is detected with an optical detector. The present invention also provides a microfluidic channel and a microfluidic cartridge for use in such a method.

### FIELD OF THE INVENTION

The present invention relates to methods for detecting an analyte present in a sample. The present invention also provides a microfluidic cartridge for use in such a method, as well as a matrix for specific use in such a method or cartridge.

### BACKGROUND TO THE INVENTION

Tests on fluid samples, such as blood are carried out for a variety of reasons. It is also desirable to be able to test samples of whole blood, rather than say plasma samples. However, constituents in the blood, such as red blood cells, can interfere or reduce the efficiency of detection. For example, tests based on conducting optical detection of an enzyme activity or analyte present in the sample of blood can be difficult as the blood itself can interfere with the optical detection. This can be resolved by removing the enzyme/analyte from the bulk of the sample of blood, but this is not always possible, particularly when the method relies on the sample of blood being present. This is particularly the case where, for example, the effect of an enzyme which is present in the sample of blood has to be determined over a period of time.

One such example is the prothrombin (PT) or international normalised ratio (INR) test. PT or INR tests are often used to check an individual's coagulation status. This is particularly the case for individuals who are taking oral anticoagulant therapy, such as warfarin or coumarin, where appropriate control of an individual's coagulation status is of extreme importance.

There are a variety of methods including electrochemical, mechanical and optical based methods for determining PT/INR values. One such test is the CoaguChek® S system (Roche) which employs an optical based system to detect blood flow in a reagent cartridge. A monitor detects the time from sample application to cessation of blood flow (clotting) and converts this into a PT. A more recent test is the CoaguChek® XS system (Roche); this employs an electrochemical based system to detect blood flow in a reagent cartridge. However, significant and regular controls are required to ensure accuracy and there is a limitation in the range of INR values which can be detected.

Other systems, including the i-STAT system of Abbott utilise electrochemical or optical sensor within the test cartridge itself, in order to avoid any optical interference by the blood.

U.S. Pat. No. 5,059,525 describes a system for use in PT tests which employs a test strip comprising suitable reagents dried on carrier material. However, in order to detect a suitable colour change it appears necessary to separate plasma from the blood, otherwise red blood cells are likely to interfere with, or obscure the colour change.

Another optical method employs the use of a rhodamine-110-based fluorescent thrombin substrate provided in a permeable membrane, having an application face and an indicator face in lateral opposition. Fluorescence kinetics are analysed in order to provide a prothrombin time which can be converted to an INR (Zweig S E, et al, Biomed Instrum Technol. 1996 May-June; 30(3):245-56.)

### SUMMARY OF THE INVENTION

The present teaching is based on studies by the inventors into developing optical detection based assays which can be conducted in a microfluidic system comprising one or more microfluidic channels along which a sample, for example whole blood, can be transported and an optical change detected using a separate optical detector positioned adjacent to said microfluidic channel. Moreover, more generally the inventors sought to mitigate or overcome the possibility of one or more constituents in a sample interfering with or obscuring optical detection.

In its broadest sense the present teaching is based on the development of a microfluidics based system/method in which optical detection of a suitable analyte or reaction product thereof within a microfluidic channel is achieved by ensuring that the means for optical detection, present within a separate reader device and the suitable analyte or reaction product thereof for detection, are in close proximity. This ensures minimising any effect that constituents (for example cells or particulate material), which may be present in the sample, may have a potential in interfering with and/or obscuring optical detection otherwise.

It will be appreciated that any of the features described herein (including any accompanying claims and drawings), may be combined with any of the below aspects in any combination, unless otherwise indicated.

In a first aspect there is provided a kinetic assay method for use in detecting an analyte within a sample, the method comprising:
1) providing a sample to a detection zone of a microfluidic channel, the detection zone comprising an optically transmissible portion and a reagent(s) localised to an inner luminal surface of the optically transmissible portion of the microfluidic channel, wherein the reagent(s) is/are associated with a porous matrix and is/are capable of reacting with the analyte or a reaction product thereof to form a reagent reaction product, the reagent reaction product capable of being detected, using an optical detector which is extra luminal to the optically transmissible portion of the microfluidic channel;
2) taking at least one optical measurement of the reagent reaction product through the optically transmissible portion; and
   detecting any analyte, based upon the at least one optical measurement of the reagent reaction product.

By “reaction product”, this will be understood to mean the product of a reaction involving the analyte or reagent, as appropriate. Reaction will thus be understood by the skilled person in the chemical sense. The reaction transforms or changes the analyte or reagent to result in the reaction product, such that the reaction product, while resulting from the analyte or reagent, is different or transformed from the analyte or reagent. The reaction product will thus be understood to be a changed entity from the analyte or reagent as the result of a reaction. In some embodiments the reaction product is an altered form of the analyte or reagent.

The reagent(s) may be adhered, bound or otherwise localised to the inner luminal surface by virtue of being associated with the porous matrix which is adhered, bound or otherwise localised to the inner luminal surface.

As used herein “matrix” will be understood to refer to a support or supports in or on which one or more components, for example the reagent or the reagent reaction product thereof, can be retained, bound or otherwise contained. This advantageously localises the reagent and any reagent reaction product thereof such that it is in close proximity to the optical detector. Any further reagent(s) necessary for the assay may also be contained within and/or on the matrix.

Optical measurement of the reagent reaction product may occur within, at the periphery and/or outside the matrix. In one embodiment, the optically detectable signal is generated within and/or at the periphery of the matrix.

Conveniently, the reagent reaction product is optically detected. For example, a change in optical signal may be generated upon generation of the reagent reaction product. Thus, the reagent reaction product may be optically detected by way of a change in the optical signal being generated. The change in optical signal may be, for example, a change in fluorescence intensity or lifetime. An optical signal may only be capable of being emitted upon formation of the reagent reaction product from the reagent. This provides a clear change in signal.

The matrix is a porous matrix. Typically the reagent(s) are mixed with material forming the porous matrix, in order that the reagent(s) are dispersed throughout the porous matrix. The porous matrix in the detection zone may be referred to herein as the porous matrix for generating an optical signal.

The skilled person will understand the term “porous” to mean having a porous network or being permeable. The present inventors believe that the porous nature of the porous matrix allows ingress of necessary analytes, for example enzymes, or reaction products thereof, from the sample, into and/or on the porous matrix. This permits any analyte or reaction product thereof to come into contact and react with the reagent(s) present within and/or on the matrix, such that following reaction any reagent reaction product thereof is in close proximity to the optical detector. Without wishing to be bound by theory, the inventors envisage that the porous matrix can be provided such that is generally impermeable to constituents of a large size such as red blood cells or other particulate material which may be present within a sample, which could interfere with and/or obscure optical detection. Thus, the porous matrix may act as a filter and may not permit the ingress of constituents which could interfere with and/or obscure optical detection.

It will be understood that detection may occur at or adjacent the site of the optically transmissible portion, i.e. the optical detector may be positioned adjacent to the optically transmissible portion, in order to detect an optical signal through the optically transmissible portion. Alternatively, the optical detector need not be adjacent to the optically transmissible portion, but any optical signal may be detected and transmitted to the optical detector which is not adjacent to the optically transmissible portion. For example an optical fibre or fibres may be positioned adjacent to the optically transmissible portion in order to detect a signal and the optical fibre or fibres are capable of transmitting the optical signal, detected through the optically transmissible portion, to the optical detector. In some embodiments, the optical signal may be detected through the use of a camera and suitable optics which are designed to detect or enable detection of a change in optical signal.

In the context of the present invention, microfluidic will be understood to mean a channel capable of containing a microfluidic volume, i.e. a volume of microlitres, for example 10 μl or less. However, it will be appreciated that in some embodiments the term microfluidic is taken to include channels which are capable of conveying volumes of less than 1 μl, i.e. nanofluidic volumes such as 10 nl-100 nl or greater.

The reagent reaction product thereof is capable of being directly or indirectly detected optically within and/or at the periphery of the porous matrix. The localised nature of the porous matrix advantageously localises a direct or indirect optical signal of the reagent or the reagent reaction product thereof. The localised nature of the signal ensures that the optical signal can be detected even when only low levels of the analyte are present in the sample.

By “within the porous matrix” it will be understood that this refers to in the porous matrix and/or on a surface of the porous matrix. The matrix may be generally porous to the liquid sample and/or reagents/analytes which are present in the sample, such as dissolved reagents/analytes, However, the matrix may not be porous to suspended material which may be present in the sample, such as cells, or other suspended material. Thus, for example, when the sample is a sample of whole blood, a liquid component of blood, with small molecules and/or proteins dissolved or suspended therein, may be able to enter the porous matrix, but larger blood components, such as cells and cellular material, such as cell fragments and platelets, may not be able to enter the porous matrix due to the size of such larger blood components.

More than one enzymatic and/or reaction step may be required in order to generate a desired optical signal. For example, two, three or more steps may be necessary depending on the analyte to be detected.

When two or more steps are envisioned, the necessary reagents may be present in a single porous matrix (the porous matrix for generating an optical change/signal) or the necessary reagents may be present in two or more porous matrices, or at least some of the necessary reagents may be present outside the porous matrix for generating the optical change/signal. Thus, multiple reaction steps may occur within a single porous matrix, or more than one porous matrix may be provided with different reagents, or at least some reagents provided outside the porous matrix for generating the optical change/signal, in order that different reactions may take place at different locations with the microfluidic channel. For example, in one embodiment, two porous matrices may be provided, the first porous matrix may comprise the necessary reagents in order to generate an analyte reaction product and the second porous matrix may comprise the necessary reagent(s) capable of generating a detectable optical change/signal due to formation of the reagent reaction product on contact with the analyte reaction product (i.e. the porous matrix for generating an optical change/signal). Thus, in accordance with the invention, it is possible to separate or de-couple the reactions necessary to generate the optical signal to be detected. This may be advantageous where reagents, constituents or excipients within a single porous matrix may, for example, interfere or otherwise with one another.

When two or more porous matrices are provided, or some reagents are separated from the porous matrix for generating the optical change/signal, it is envisaged that at least the porous matrix for generating an optical change/signal is localised to an inner luminal surface of the optically transmissible portion of the detection zone. The additional porous matrix/matrices or the separated reagents may also be localised to an inner luminal surface of the optically transmissible portion of the detection zone. Alternatively, the additional porous matrix/matrices, or separated reagents may be localised to a section of the microfluidic channel which is not the detection zone. This conveniently separates some reactions from the detection zone and thus may help to reduce or prevent interference of optical detection.

As mentioned above, in use the optically transmissible portion is intended to be positioned so as to be in close proximity with the optical detector or optical detection component thereof, in order to ensure efficient detection and/or reduce any interference which may occur due to components in the sample, such as red blood cells in a sample of whole blood. In some embodiments the optically transmissible portion of the channel is designed to be a top (or “upper”) portion of the channel. It will be appreciated that the term “top” or “upper” refers to the optically transmissible portion when in use and so the skilled reader will readily appreciate and understand its meaning. The optically transmissible portion should be positioned to be adjacent to or on the same side as the optical detector or optical detection component thereof. Conveniently when the optical detector or optical detection component thereof is directed towards the top portion, the detector or component thereof will be pointing downwards. Advantageously, this may limit dust build up on the detector or component thereof. Optical detection is thus not obscured. This advantage especially applies to embodiments where the porous matrix is localised to an upper luminal surface. Thus, in some embodiments the porous matrix is localised to an upper inner luminal surface of the optically transmissible portion. Thus, the porous matrix is held against the force of gravity. Without wishing to be bound by theory, the present inventors believe that by localising the porous matrix to the upper inner luminal surface of the optically transmissible portion ensures that constituents of the sample which could otherwise interfere with detection, for example, red blood cells are kept outside the porous matrix. As a result, interference with optical detection is reduced or removed. However, the porous matrix may in other embodiments be localised to a lower (or “bottom”) inner luminal surface of the optically transmissible portion, in which case the optical detector or optical detection component thereof will be positioned below the optically transmissible portion. In essence the invention is such that the sample is not intended to come between the porous matrix and the optical detector or optical detection component thereof and hence does not interfere or otherwise occlude any optical signal generation within the porous matrix and hence its detection.

In embodiments comprising additional porous matrix/matrices, the additional porous matrix/matrices may be localised to an inner luminal surface of a section of the microfluidic channel which is not the detection zone. The inner luminal surface may be an upper or a lower luminal surface.

The present invention relates to kinetic assays, sometimes called end-point assays. Kinetic assays are enzyme-based assays that measure the amount of a material or analyte by the quantity of a substrate consumed or product formed over the course of a reaction. However, it is not necessary for a formal end-point to be reached and in many instances it is a rate of reaction which is determined.

The present invention is suitable, for example, with any endpoint or kinetic assay which may be conducted on a sample. As the skilled person will appreciate, an endpoint assay comprises taking one measurement after a fixed period of time. Hence, step 2) may be performed after a fixed period of time.

The period of time may be at least 10 seconds, at least 30 seconds, at least 1 minute, at least 1.5 minutes, at least 2 minutes, at least 3 minutes or at least 5 or 10 minutes. The period of time may be no more than 10 or 5 minutes, no more than 3 minutes, no more than 2 minutes, no more than 1.5 minutes or no more than 1 minute. In some embodiments the period of time is approximately 2 minutes.

In accordance with an embodiment of the invention, step 2) may comprise taking a plurality of optical measurements of the reagent reaction product over a period of time, followed by a step comprising detecting any analyte based upon the plurality of optical measurements. In this manner the series of optical measurements may follow a reaction as it progresses form a substrate to a product. Typically there may be a shift in an optical signal, such as absorbance, fluorescence intensity or the like which may be detected. A spectrophotometer or fluorimeter may be used, for example, to monitor any change in optical signal, which may be an increase or decrease in signal over time.

Typically such assays may be of use in detecting or determining an amount of one or more analytes in the sample. Where the sample is a sample of blood or other bodily fluid, the analyte may include, for example, an enzyme, lipid, lipoprotein, cytokine, hormone or other biomarker which may be present in the sample, including endotoxins and the like. Typically, analytes may comprise an enzyme, such as a blood clotting factor and/or lipid, such as cholesterol or other lipoprotein such as lipoprotein (a).

The inventors generally envisage the assay as an enzymatic or lipid/lipoprotein assay. In an enzymatic assay the analyte comprises one or more enzymes. In a lipid/lipoprotein assay the analyte comprises one or more lipids/lipoproteins.

The reagent reaction product will be understood to be a product resulting from a reaction of the analyte or the analyte reaction product thereof with the reagent.

The reagent reaction product may be as a result of a direct reaction between the analyte and a reagent. For example, in embodiments comprising enzymatic assays, the reagent reaction product may be derived from an analyte which has directly reacted with the reagent to form the reagent reaction product. Reaction may comprise contact, for example cleavage of the analyte by the enzyme to form the reagent reaction product, or the analyte comprises the enzyme which cleaves the reagent to form the reagent reaction product. Alternatively, reaction may comprise or consist of oxidisation or reduction of the analyte by the reagent, or vice versa, to form an oxidised or reduced reagent reaction product.

In some embodiments the reagent reaction product is as a result of an indirect reaction between the analyte and a precursor to the reaction product. For example, the analyte may directly react with a component of a pathway, to form an analyte reaction product. The analyte reaction product may then react with a downstream component (which in the context of the present invention may be understood as a further analyte reaction product) of the pathway. The analyte reaction product, further analyte reaction product or a further downstream component of the pathway may then directly react with a reagent to form the reagent reaction product, thereby generating the reagent reaction product.

One area where assays in accordance with the present invention may be used is in wound care/healing. Here proteases such as matrix metalloproteinases may be monitored for example (see Lazaro, J. L. et al, J of Wound Care, Vol 25, No. 5, May 2016).

Enzymes for detection include enzymes of the contact activation (intrinsic) and tissue activation (extrinsic) pathways and other enzymes such as proteases or nucleases which may be present in blood. In one embodiment the present invention is directed to the detection of one or more enzymes of the extrinsic or intrinsic pathway, or its common pathway components. The remaining description directed to these pathways will be described in relation to the detection of thrombin activity and hence the determination of a PT and/or INR value. However, this should not be construed as limiting and it is readily possible to envisage methods which are capable of detecting and/or determining the effect of other members of the extrinsic or intrinsic pathway, or enzymes from other pathways.

Other members of the extrinsic or intrinsic pathway include, but are not limited to Factor X, Factor Xa, Factor II, fibrin monomer and protein C.

In some embodiments the analyte for detection comprises thrombin. The method may therefore comprise detecting any thrombin, based upon the at least one optical measurement of the reagent reaction product.

The detected thrombin may be used to determine a prothrombin time (PT) or international normalized ratio (INR) value for the sample. For example, the porous matrix may comprise a thrombin cleavable substrate as the reagent, which is capable of generating a detectable optical signal, for example a fluorescent signal, upon cleavage by thrombin. The thrombin cleavable substrate may comprise a peptide sequence which is recognisable and cleavable by thrombin and an associated fluorescent molecule which is capable of detection following cleavage of the peptide. This will be discussed in more detail hereinafter. Typically, the thrombin cleavage product is retained within and/or remains adjacent, or at the periphery of the porous matrix.

Lipids and/or lipoproteins for detection include, but may not be limited to, lipoprotein (a), chylomicrons, very low density lipoproteins (VLDL), intermediate-density lipoproteins (IDL), phospholipids, cholesterol and triglycerides. As the skilled person will appreciate, cholesterol may comprise low-density lipoprotein (LDL) cholesterol and high-density lipoprotein (HDL) cholesterol. The cholesterol may comprise free cholesterol and/or cholesteryl esters.

In some embodiments the analyte for detection comprises cholesterol.

As the skilled person will appreciate, when cholesterol is in the presence of the enzyme cholesterol oxidase, the following chemical reaction occurs:

cholesterol+O₂⇄cholest-4-en-3-one+H₂O₂

Hence, the sample may comprise endogenous cholesterol oxidase, and/or exogenous cholesterol oxidase may be added to the sample or be present in the porous matrix. In some embodiments the cholesterol reaction product is the analyte for detection.

The cholesterol reaction product may comprise hydrogen peroxide. In some embodiments hydrogen peroxide oxidises the reagent to form the reagent reaction product.

The sample may be a biological sample. Conveniently the biological sample may be any appropriate fluid or tissue sample obtained from a subject. For example, the biological sample may comprise at least one of urine, saliva, whole blood, plasma, sputum, semen, faeces, a nasal swab, a wound swab, tears, a vaginal swab, a rectal swab, a cervical smear, a tissue biopsy, and a urethral swab. Suitably, the biological sample is one that can be readily obtained from a subject, such as urine, saliva, whole blood and sputum, which the subject may be able to collect from him/herself, without the need for assistance or any invasive surgical technique. In some embodiments the sample is whole blood or a wound swab. In one embodiment, the sample is whole blood.

The method may further comprise a preliminary step, comprising introducing the sample to the microfluidic channel and thereafter transporting at least a portion of the sample to the detection zone. The sample may be introduced via an inlet port which may be connected to the microfluidic channel. The sample may be introduced directly by contacting the sample with the input port. Alternatively, the sample may first be collected using a sample collection means and such sample collection means, such as a dipstick, micropipette, capillary tube or the like, contacted with or inserted into the sample input port in order that the sample may be introduced into the microfluidic channel.

In some embodiments, such as when carrying out nucleic acid analysis, it may be desirable to carry out the method in a closed system. Thus, the sample collection means designed to introduce the sample into the microfluidic channel may serve the dual purpose of introducing the sample and sealing the sample input port, once the sample collection means has been inserted into/contacted/with the sample input port.

In a further aspect there is provided a microfluidic channel for use in a method as described herein, the microfluidic channel comprising:

a detection zone for receiving at least a portion of a sample provided to the microfluidic channel, the detection zone comprising an optically transmissible portion and a porous matrix comprising a reagent localised to an inner luminal surface of the optically transmissible portion of the microfluidic channel, wherein the reagent is capable of reacting with an analyte in the sample to be detected, or an analyte reaction product thereof, in order to generate a reagent reaction product which is capable of being optically detected.

The microfluidic channel may be formed from three layers; a top and a bottom layer with a middle layer disposed between the top and bottom layers, which are sandwiched together to define the microfluidic channel. Hence, the side walls of the microfluidic channel may be defined by the middle layer. In some embodiments the top layer comprises the optically transmissible portion of the channel. The porous matrix may be deposited on the luminal surface of the top layer. Only the portion of the top layer which is intended to overlay the detection zone may be optically transmissible. In some embodiments the top layer consists of the optically transmissible portion. In some embodiments the top layer consists of the optically transmissible portion and the porous matrix for generating an optical change/signal is deposited on the luminal surface of the top layer. In some embodiments the porous matrix for generating an optical change/signal is deposited on the luminal surface of the bottom layer.

In some embodiments the middle layer is in the form of an adhesive layer which adheres the top and bottom layers, thereby forming a sandwich. Further details of suitable microfluidic channels and their presence in suitable microfluidic cartridges may be found in WO 2018/002668 to which the skilled reader is directed and the entire contents of which are hereby incorporated.

By localising the porous matrix comprising the reagent to the inner luminal surface of the optically transmissible portion of the channel, the reagent reaction product can be brought into close proximity with an optical detector or optical detection component thereof present within an associated reader, which serves to facilitate detection of the reagent reaction product and/or increases the sensitivity of detection. Other constituents within the sample may not enter the porous matrix and/or are not held or otherwise remain within the porous matrix/localised to the inner luminal surface. Interference by the other constituents is thus reduced or removed. Thus, the reagent reaction product being in close proximity to the optical detector or optical detection component thereof enables detection to take place in an environment where constituents are less able to interfere and affect the optical detection. In this manner, the optical detection of the reagent reaction product can take place without constituents, for example red blood cells, generally being in the light path between the optical detection device and the reagent reaction product. Conventionally, if a reagent reaction product was dispersed throughout the microfluidic channel, then constituents (such as red blood cells and other cellular material in whole blood) are likely to be in the light path between the optical detection means and the reagent reaction product, leading to interference and/or a reduction in sensitivity.

Typically, although not exclusively, the microfluidic channel may be comprised within a cartridge. In some embodiments a plurality of microfluidic channels are provided. Two, three, four, five, six, seven, eight, nine, ten or more microfluidic channels may be provided within the cartridge.

Optionally, when present, the inlet port is also comprised within the cartridge. The cartridge may be designed to be inserted into an associated reader device. For brevity, hereinafter reference will be made to the microfluidic channel being present within a cartridge, but this is not to be construed as limiting.

In a further aspect there is provided a microfluidic cartridge for use in conducting an assay of the first aspect on a sample, the microfluidic cartridge comprising:

at least one microfluidic channel, wherein each/said microfluidic channel(s) comprises a detection zone, the detection zone comprising an optically transmissible portion and a porous matrix comprising a reagent localised to an inner luminal surface of the optically transmissible portion, wherein the reagent is capable of reacting with an analyte or reaction product in the sample to be detected in order to generate a reagent reaction product which is capable of being optically detected.

The system may be formed from three layers, which are sandwiched together to define each/said microfluidic channel(s).

Optionally, each/said microfluidic channel(s) is further fluidly connected to an externally compressible, gas-filled chamber downstream from each/said detection zone, wherein compressing or decompressing said chamber, using compression means outside the chamber, causes gas to be expelled from or drawn into the chamber, which in turn causes movement of the sample within said/each microfluidic channel. Further details of suitable microfluidic cartridges which include gas-filled chambers and how they function, may be found in WO 2018/002668 to which the skilled reader is directed and the entire contents of which are hereby incorporated.

The microfluidic cartridge will typically further comprise a sample entry/input port, in fluid communication with each/said microfluidic channel. In some embodiments the sample entry port is in the form of a well into which a sample may be placed. In certain embodiments, the well may be generally circular in shape and is sized such that a drop of blood may be easily received into the well. The well may have a volume of at least 1 μl and no more than 30 μl. The well may have a volume of at least 4 μl and no more than 10 μl. The well opening may be formed in the top surface of the cartridge. Typically the total volume of sample, such as blood, which is drawn into the cartridge and fills up to and including the detection zone may be between 3-6 μl, such as between 3.5-5 μl. The total volume of the detection zone may be in the region of 0.75 μl-1.25 μl. Thus, the assays/methods of the present invention are capable of analysing extremely small volumes of sample, which is advantageous.

The sample entry or input port may generally be located on the top surface of the cartridge, so a sample may easily be applied and introduced into the cartridge and transported within the microfluidic channel. It will be appreciated therefore than in an embodiment the input port is located on the same substrate to which the porous matrix may be adhered. The input port permits entry of a sample to the lumen of the microfluidic channel from an outer top surface of the cartridge and the porous matrix is adhered to an inner surface within the lumen of the microfluidic channel.

In one embodiment the optically transmissible portion is provided on the top surface or face of the microfluidic cartridge.

For the avoidance of doubt the microfluidic channels or cartridges of the present invention do not require the use of liquid-filled pouches to be present within or provided with the cartridge, moving parts, e.g. a piston, within the chamber and/or the ability to transfer fluid (liquid or gas) from an associated reader to the cartridge. In this regard the cartridges of the present invention may be considered as being self-contained. The cartridges of the present invention prior to application of a sample are substantially or completely liquid free and may be considered as dry. The only fluid prior to application of the sample, which may be, or is present in the cartridge, will be a gas, typically air. Advantageously the only liquid required in the assays of the present invention, is the sample itself.

The substantially or completely liquid-free nature of the cartridge not only simplifies manufacturing of the cartridges themselves, but also improves shelf-life and allows many of the cartridges of the present invention to be stored at room temperature, with little degradation of the chemical or biological components within the cartridge, prior to use.

In some embodiments, the method further comprises a step of inserting the cartridge into a reader device. The step of inserting the cartridge into the reader may generally occur before application of the sample to the cartridge/microfluidic channel. The reader device comprises the optical detector or optical detection component thereof. Further details of suitable reader devices may be found in WO 2018/002668 to which the skilled reader is directed and the entire contents of which are hereby incorporated.

In some embodiments, the method comprises at least two preliminary steps comprising:
a) inserting the cartridge into a reader device; and
b) introducing the sample to each/said microfluidic channel.

Introduction of the sample to each/said microfluidic channel may occur by passive means (e.g. capillary action), by active means (e.g. by application of a motive force, such as by using a pump), or by a mixture of the two. For example, in one embodiment, the sample is initially introduced into the microfluidic channel by capillary action and thereafter further sample movement is controlled by an active force, such as by use of a pump. The pump will typically be external to the cartridge, for example in an associated reader device.

The sample may be provided to the detection zone by an active fill mechanism. In some embodiments movement of the sample into and/or through the detection zone and/or by a porous matrix is by active means.

Typically, the cartridge may be provided or inserted into the reader device prior to sample application.

By optically transmissible, it will be understood that light of a defined wavelength can be transmitted and received through the portion, i.e. the portion does not substantially quench or absorb wavelengths of light transmitted and received through the portion. In this way light from the optical detector can be transmitted through the portion to meet the analyte or analyte reaction product which in turn is reflected back or absorbed and re-emitted and can be detected by the optical detector. In some embodiments the portion is transparent. In some embodiments the portion is translucent. By defined wavelength, this will be understood to include, but not necessarily be limited to, wavelengths of at least 10 nm and no more than 1500 nm. Typically, the wavelength of light from the optical detection device transmitted and received through the optically transmissible portion may be at least 300 nm and no more than 800 nm.

The porous matrix or matrices and optionally separated reagents/components may be localised to the inner luminal surface, such as an upper inner surface by binding to the inner luminal surface. The binding may be direct or indirect, for example, by way of physical adsorption, covalent chemical coupling, a scaffold, non-covalent chemical bonding (e.g. biotin-avidin) or a combination of any of the above. In one embodiment the porous matrix is physically absorbed not through any covalent or ionic bonding. A precursor to an analyte reaction product (i.e. a component of a reaction with the analyte) may be localised to the inner luminal surface and/or provided as part of the porous matrix and/or within or on the porous matrix. The precursor may be capable of interacting with the analyte in order to generate an analyte reaction product. For example, the precursor may be capable of binding and/or reacting with the analyte. The porous matrix may therefore act as a scaffold for the precursor.

Conveniently, the porous matrix for generating an optical change/signal is viscous and so can withstand fluid/blood flow. Any additional matrices may also be viscous, although in some embodiments an additional matrix is less viscous than the porous matrix for generating an optical change/signal. Accordingly, the porous matrix and, where present, associated precursor and/or reagent remain aligned or in close proximity with the optical detector to ensure optimal detection after contact with the sample.

It will be understood that the porous matrix for generating an optical change/signal is, or may be partially, optically transmissible. In embodiments comprising one or more additional matrices, the additional matrix/matrices may also be at least partially optically transmissible. In some embodiments the porous matrix is transparent. In some embodiments the porous matrix is translucent. This may facilitate with detection of an optical signal from within and/or on the porous matrix.

The porous matrix may comprise at least one carrier molecule, such as a polymer. In one embodiment the carrier molecule is generally insoluble in aqueous based solutions. The at least one carrier molecule may be generally insoluble in the sample fluid, at least for the period of time required for the assay to be conducted. In aqueous based solutions, the carrier molecule may form a colloid. “Colloid” will be understood to refer to a carrier molecule which is insoluble but is capable of being suspended/dispersed throughout a solvent. The solvent may be water. In one embodiment the carrier molecule is not generally soluble in blood. In some embodiments, the carrier molecule comprises a kosmotrope. By “kosmotrope”, it will be understood that the carrier molecule causes water molecules to favourably interact, i.e. the polymer contributes to the stability and structure of water-water interactions.

In some embodiments the at least one carrier molecule comprises at least one carbohydrate. The at least one carbohydrate may comprise at least one monosaccharide, disaccharide, or polysaccharide. As the skilled person will appreciate, a polysaccharide is a polymer.

In some embodiments the at least one carrier molecule comprises at least one sugar. In the context of the present invention, sugar will be understood to refer to monosaccharides and disaccharides.

In some embodiments the at least one carrier molecule comprises at least one disaccharide or polysaccharide.

Example monosaccharides include, but are not limited to glucose, fructose, galactose, triose, tetrose, pentose, hexose and heptose.

Example disaccharides include, but are not limited to, sucrose, lactose, maltose, trehalose, cellobiose and chitobiose.

Example polysaccharides include, but are not limited to amylose, amylopectin, cellulose, chitin, callose, laminarin, chrysolaminarin, xylan, arabinoxylan, mannan, fucoidan and galactomannan.

In particular embodiments, the at least one carrier molecule comprises at least one disaccharide.

Preferably, the at least one carrier molecule comprises at least trehalose.

In some embodiments, the at least one carrier molecule comprises at least one polysaccharide. In some embodiments the at least one polymer comprises cellulose or chitin. In other embodiments, the at least one polymer comprises at least one cellulose derivative.

In some embodiments the at least one carrier molecule comprises cellulose or at least one cellulose derivative

In some embodiments the at least one carrier molecule comprises trehalose and at least one cellulose derivative.

The cellulose derivative may be selected from the group consisting of carboxymethylcellulose (CMC), cellulose ethyl sulfonate (CES), hydroxyethylcellulose (HEC), hydroxypropyl methyl cellulose (HPMC), hydroxypropyl cellulose (HPC), microcrystalline cellulose (MCC), methylcellulose and salts thereof, for example sodium, potassium, calcium salts.

A preferred salt may be sodium.

The cellulose derivative may be selected from the group consisting of carboxymethylcellulose (CMC), hydroxyethylcellulose (HEC), microcrystalline cellulose (MCC) and salts thereof.

In some embodiments the cellulose derivative is selected from the group consisting of carboxymethylcellulose (CMC), hydroxyethylcellulose, microcrystalline cellulose and sodium carboxymethylcellulose.

The cellulose derivative may comprise carboxymethylcellulose and microcrystalline cellulose, for example the cellulose derivative Avicel RC-591

The cellulose derivative may be carboxymethylcellulose (CMC).

The cellulose derivative may be hydroxyethylcellulose (HEC).

In some embodiments the cellulose derivative comprises a degree of substitution of at least 0.1, at least 0.2, at least 0.3, at least 0.4, at least 0.5, at least 0.6, at least 0.7, at least 0.8, at least 0.9, at least 1.1, at least 1.2, at least 1.5, at least 2 or at least 3. For example, in the context of carboxymethylcellulose, the degree of substitution is the average level of methoxy (carboxymethyl) substitution on the cellulose chain (i.e. the average number of hydroxyl groups substituted for methoxy per anhydroglucose unit.). Hence, in the context of carboxymethylcellulose, a degree of substitution value of 0.7 will be understood to refer to an average of 7 carboxylmethyl groups per 10 anhydrogluocose units, while a degree of substitution value of 3 will be understood to mean all three hydroxyls on the anhydroglucose unit being replaced with carboxymethyl groups.

In some embodiments the cellulose derivative comprises a degree of substitution of at least 0.5 and no more than 2. In some embodiments the cellulose derivative comprises a degree of substitution of at least 0.5 and no more than 1.2. In some embodiments the cellulose derivative comprises a degree of substitution of at least 0.7 and no more than 1.2.

In a particular embodiment the cellulose derivative comprises CMC and comprises a degree of substitution of at least 0.7 and no more than 1.2.

The cellulose may be crystalline.

In some embodiments the at least one carrier molecule comprises polyethylene glycol (PEG).

The porous matrix may comprise at least two, at least three, at least four, at least five, at least six, at least seven, at least eight, at least nine, or at least 10 carrier molecules.

In some embodiments the porous matrix comprises no more than 10, no more than nine, no more than eight, no more than seven, no more than six, no more than five, no more than four, no more than three or no more than two carrier molecules.

In particular embodiments the porous matrix comprises at least two carrier molecules. In some embodiments the at least two carrier molecules comprises at least two carbohydrates. The at least two carrier molecules may comprise at least one disaccharide and cellulose or a cellulose derivative. The disaccharide may comprise trehalose. The at least two carrier molecules may comprise at least one carbohydrate and polyethylene glycol.

In a particular embodiment, the at least two carrier molecules comprises trehalose and at least one cellulose derivative. The cellulose derivative may be carboxymethylcellulose (CMC).

In some embodiments, the at least two carrier molecules comprises at least cellulose and sodium carboxymethylcellulose.

In particular embodiments, the at least two carrier molecules comprises trehalose and carboxymethylcellulose.

In some embodiments, the at least two carrier molecules comprises hydroxyethylcellulose (HEC) and trehalose.

In other embodiments, the at least two carrier molecules comprises cellulose and sodium carboxymethylcellulose.

In other embodiments the porous matrix comprises at least three carrier molecules. The at least three carrier molecules may comprise at least three carbohydrates. The at least three carrier molecules may comprise at least one disaccharide and cellulose. In some embodiments the at least three carrier molecules comprises at least one disaccharide and at least one cellulose derivative. In some embodiments the at least three carrier molecules comprises at least one disaccharide, cellulose and at least one cellulose derivative. In other embodiments the at least three carrier molecules comprises at least one disaccharide, cellulose or a cellulose derivative and PEG.

In some embodiments, the at least three carrier molecules comprises at least cellulose, sodium carboxymethylcellulose and trehalose.

In some embodiments, the at least three carrier molecules comprises hydroxyethylcellulose (HEC), trehalose and PEG.

In some embodiments, the at least three carrier molecules comprises cellulose, sodium carboxymethylcellulose and trehalose.

In other embodiments, the at least three carrier molecules comprises cellulose, sodium carboxymethylcellulose and HEC.

In some embodiments the porous matrix comprises at least four carrier molecules. The at least four carrier molecules may comprise at least four carbohydrates. The at least four carrier molecules may comprise at least one disaccharide, cellulose or a cellulose derivative and PEG. In some embodiments the at least four carrier molecules comprises at least one disaccharide, cellulose, at least one cellulose derivative and PEG.

In other embodiments, the at least one carrier molecule comprises hydroxyethylcellulose (HEC).

The porous matrix may initially be provided in a liquid form which is allowed to dry by evaporation or other means. Hence, reference to concentration in the matrix, as used herein, refers to the concentration of the molecule/component in the porous matrix in a liquid form (i.e. before the matrix is allowed to dry by evaporation or other means and before sample is added).

The liquid form of the porous matrix may comprise a volume of at least 50 nl and no more than 200 nl, preferably at least 100 nl and no more than 160 nl. In this manner it can be appreciated that the deposited porous matrix even before drying takes up a relatively small volume of the total volume of the detection zone. For example, the porous matrix, prior to drying may comprise a volume less than 40%, 30%, or 25%, of the total volume of the detection zone.

The at least one carrier molecule may be provided at a concentration of at least 0.05 w/v % and no more than 40 w/v % in the porous matrix. Typically, the at least one carrier molecule may be provided at a concentration of at least 10 w/v % and no more than 15 w/v %.

By concentration of the carrier molecule, it will be understood that this is the final concentration of the carrier molecule in the porous matrix when wet (i.e. the matrix is applied as a liquid form which is allowed to dry by evaporation or other means before sample is added). Hence, where the carrier molecule comprises more than one carrier molecule, the final concentration will be understood to be the final concentration of all of the carrier molecules.

In particular embodiments, the at least one carrier molecule comprises at least trehalose. Trehalose may be provided at a concentration of at least 5 w/v %, at least 6 w/v %, at least 8 w/v %, at least 10 w/v %, and of no more than 30 w/v % or no more than 40 w/v %.

By concentration of trehalose, it will be understood that this refers to the final concentration of the trehalose in the porous matrix when applied as a liquid form, and does not refer to the final concentration of the carrier molecule when the carrier molecule comprises carrier molecules additional to trehalose.

In some embodiments trehalose is provided at a concentration of approximately 10 w/v %.

In particular embodiments, the at least one carrier molecule comprises at least carboxymethylcellulose (CMC). CMC may be provided in the porous matrix at a concentration of at least 0.05 w/v % and no more than 0.6 w/v %. In some embodiments CMC is provided in the porous matrix at a concentration of at least 0.08 w/v % and no more than 0.4 w/v %.

In particular embodiments the at least one carrier molecule comprises at least HEC. HEC may be provided in the porous matrix at a concentration of at least 0.1 w/v % and no more than 2 w/v %. In some embodiments HEC is provided in the porous matrix at a concentration of at least 0.25 w/v % and no more than 1 w/v %.

The porous matrix may comprise HEC at a molecular weight of at least 50,000 g/mol and no more than 1,300,000 g/mol. Typically, the porous matrix may comprise HEC at a molecular weight of 720,000 g/mol.

The porous matrix may comprise HEC having at least two different molecular weights.

The porous matrix may further comprise one or more components. Where the sample is a sample of blood, the one or more components may include, but are not limited to, one or more tissue factors, one or more clotting factors, one or more fluorophores, anti-fibrinogen, anti-antithrombin III, cholesterol oxidase, one or more detergents and one or more anticoagulant inhibitors. The one or more components may comprise one or more further reagents.

In assays for determining a prothrombin time (PT) or international normalized ratio (INR) value, the one or more components may include, but not be limited to, one or more tissue factors, one or more clotting factors, one or more fluorophores, anti-fibrinogen, anti-antithrombin III, one or more detergents and/or one or more anticoagulant inhibitors.

In one embodiment for PT/INR determination, the carrier molecule comprises CMC and PEG. CMC may be present in an amount between 0.025-0.125 wt %, such as 0.05-0.1 wt %. PEG may be present in an amount between 0.5-4 wt %, such as 0.75-3.5 wt %.

Other components such as trehalose (2-8 wt %), readiplastin (25-40 wt %), chromogenic substrate (e.g. enzyme cleavable Rhodamine (0.05-0.75 wt %), surfactant (e.g. Tween, 0.01-0.05 wt %) and/or polybrene 0.1-0.2 mg/ml) may be present.

In assays for determining a cholesterol value, the one or more components may include, but not be limited to one or more fluorophores, horse radish peroxidase (HRP), cholesterol oxidase, cholesterol esterase and one or more detergents.

In embodiments comprising cholesterol oxidase (whether added to the sample or as a component of the porous matrix), HRP may be included in the sample or as a component of the porous matrix as a redox mediator.

The one or more fluorophores may comprise RPE (R-phycoerythrin) and/or PERCP (peridinin chlorophyll). Advantageously, the inclusion of one or more fluorophores in the porous matrix aids detection of the porous matrix prior to application of the sample.

Anti-antithrombin III acts to inhibit ATIII (anti-thrombin III). As the skilled person will appreciate, ATIII is an autoantibody against thrombin which inhibits thrombin activity. Hence, the addition of anti-antithrombin III to the porous matrix advantageously prevents the inhibition of thrombin by ATIII. This increases the signal obtained in a thrombin-based assay.

Tissue factor, also known as Factor III, is a lipoprotein initiator of the extrinsic clotting pathway. As the skilled person will appreciate, tissue factor is available commercially as RecombiPlastin 2 g (Werfen), or Readiplastin. Readiplastin is a concentrated liquid form of RecombiPlastin 2 g in which calcium has been removed. Other commercially available tissue factors will be known to the skilled person.

The one or more clotting factors may comprise one or more of calcium, Factor V/Va, Factor X/Xa, prothrombin, Factor Vila, IXa and XIa. Factor V/Va is a cofactor which forms a prothrombinase complex with Factor Xa and calcium. This complex cleaves prothrombin converting it to the active form, thrombin. Factor V/Va is modulated by thrombin, being directly activated by thrombin on a positive feedback loop and indirectly activated by thrombin through the thrombomodulin-protein C complex (with the cofactor protein S) pathway. Other clotting factors will be known to the skilled person.

The one or more clotting factors may be selected from the group consisting of calcium, Factor V/Va, Factor X/Xa, prothrombin, Factor Vila, IXa and XIa.

The porous matrix may comprise calcium at a concentration of at least 0.1 mM and no more than 25 mM. It will be appreciated that a molar or millimolar concentration relates to the concentration when provided in a liquid form, which is then allowed to dry by evaporation or other means.

The porous matrix may comprise Factor V/Va at a concentration of at least 0.1 units/ml and no more than 120 units/ml, for example at least 10 units/ml and no more than 100 units/ml.

The porous matrix may comprise Factor X/Xa at a concentration of at least 0.1 μg/ml and no more than 20 μg/ml. In some embodiments the porous matrix comprises Factor X/Xa at a concentration of at least 1 μg/ml and no more than 15 μg/ml.

The porous matrix may comprise prothrombin at a concentration of at least 0.05 mg/ml and no more than 0.25 mg/ml.

The porous matrix may comprise Factor Vila at a concentration of at least 0.1 μg/ml and no more than 50 μg/ml.

The porous matrix may comprise Factor IXa at a concentration of at least 0.1 μg/ml and no more than 50 μg/ml.

The porous matrix may comprise Factor XIa at a concentration of at least 0.1 μgird and no more than 50 μg/ml.

One example of an anticoagulant inhibitor is polybrene (hexadimethrine bromide—HMB). The anticoagulant inhibitor may be provided in the porous matrix at a concentration of at least 0.05 mg/ml and no more than 0.5 mg/ml. In particular embodiments the anticoagulant inhibitor is provided in the porous matrix at a concentration of approximately 0.25 mg/ml.

As above, it will be appreciated that concentration at units/ml, μg/ml or mg/ml relates to the concentration when provided in a liquid form, which is then allowed to dry by evaporation or other means. The liquid form of the porous matrix may be applied as a spot, for example, before being allowed to dry. The spot after application may be spread out by mechanical or other means, so as to result in a thinner and more uniform porous matrix being provided. A thinner and more disperse spot may enhance optical signal generation and/or detection. Desirably the thickness of the dried spot is controlled and may be substantially uniform in thickness. The maximum thickness of the dried spot may be 1-5 μm, such as 1-3 μm thick. Hydrophobic printed features, such as in the form of a square or rectangle, may be provided in the detection zone and the porous matrix provided and spread within the region bounded by the hydrophobic printed features. In this manner the porous matrix is designed to be located within a defined region of the detection zone. This serves to ensure that the porous matrix can be deposited in a reproducible and uniform manner.

The one or more detergent(s) may comprise Tween-20.

The reagent reaction product is capable of being directly or indirectly detected optically within or on the porous matrix. By “directly” it may be understood that the reagent reaction product is capable of emitting an optical signal, for example a fluorescent or colorimetric signal. By “indirectly”, it may be understood that the reagent reaction product is capable of reacting with a further reagent, the reagent being capable of emitting an optical signal upon reacting with the reagent reaction product thereof. Typically, the reagent reaction product is capable of being directly detected optically.

The analyte may be capable of reacting with a reagent specific for the analyte, in order to generate an analyte reaction product, the analyte reaction product in turn being capable of reacting with a further reagent, the further reagent being capable of emitting an optical signal as the reagent reaction product upon interaction with the analyte reaction product. The reagent and further reagent may be present in a single porous matrix, or separate porous matrices, for example.

The porous matrix may comprise the reagent and/or optional further reagent(s). The porous matrix, or porous matrices comprising the reagent and optional further reagent(s) may be localised in a dry or a wet state to the inner luminal surface of the optically transmissible portion, prior to conducting the methods of the present invention. Desirably, the reagent/further reagent(s) is/are comprised in the porous matrix or porous matrices, the porous matrix/matrices being in a dry state, and wetted upon coming into contact with the sample. Dry may be considered to be substantially liquid free. The reagent(s) and/or porous matrix/matrices may initially be provided in a liquid/slurry form which is allowed to dry by evaporation or other means. In terms of the present invention, when the reagent(s) and/or porous matrix/matrices are initially presented in a liquid from, which is subsequently dried, the term “dry” is to be understood as meaning that less than 10%, 5%, or 1% of the initial liquid remains after drying. The porous matrix/matrices, and optionally the reagent(s) may be provided at any pH. In embodiments directed to biological samples, the porous matrix/matrices, and optionally the reagent(s), may be provided at a pH of at least 7 and no more than 8.5. In some embodiments the porous matrix/matrices, and optionally the reagent, is provided at pH 7±0.25.

By optical signal, it will be appreciated that may be a photometric signal. The optical signal may be fluorescence signal or colour signal. In preferred embodiments the optical signal comprises a fluorescence signal.

In some embodiments, the reagent or further reagent(s) comprises a cleavable substrate. Hence, when the reagent/further reagent(s) and the analyte/analyte reaction product react, the analyte/analyte reaction product may cleave the substrate causing release of an optical signal. In embodiments where the reagent comprises a precursor (i.e. a component of an upstream pathway), the analyte may cleave the substrate from the precursor to release the analyte reaction product. The analyte reaction product can then react with the reagent to form the reagent reaction product. The analyte reaction product may be capable of emitting an optical signal. The substrate may comprise one or more enzyme cleavable peptides. In some embodiments the cleavable substrate comprises a thrombin cleavable substrate, which is capable of generating a detectable optical signal upon cleavage by thrombin. The thrombin cleavable substrate may comprise a peptide sequence which is recognisable and cleavable by thrombin and an associated fluorescent molecule which is capable of detection following cleavage of the peptide.

In embodiments, the reagent/further reagent is capable of being oxidised. Hence, in some embodiments the reagent/further reagent(s) is/are capable of being oxidised by an analyte or an analyte reaction product to form a reagent reaction product which emits an optical signal. In embodiments where the reagent/further reagent comprises a precursor, the reagent/further reagent may be capable of being oxidised by the analyte in order to form an oxidised analyte reaction product. The analyte or analyte reaction product may comprise hydrogen peroxide. For example, cholesterol oxidase can act upon the analyte cholesterol to form the analyte reaction product hydrogen peroxide. Hydrogen peroxide may be capable of oxidising the reagent to form the reagent reaction product so that the reagent emits an optical signal. The reagent may comprise horse radish peroxidase.

Detection of an optical signal may comprise detection of an emission of an optical signal, for example a fluorescent signal. In some embodiments, detection comprises detecting an alteration in optical signal, such as a change in the wavelength of light emitted from the reagent, intensity of signal and/or signal lifetime.

As the skilled person will appreciate, a fluorophore is a fluorescent compound that can re-emit light upon light excitation of an appropriate wavelength. Hence, where capable of emitting an optical signal, the analyte, analyte reaction product, reagent and/or reagent reaction product may comprise a fluorophore. The fluorophore may be provided in an inactive state (i.e. non-fluorescing).

Release of a substrate from the fluorophore may activate the fluorophore to fluoresce, generating an optical signal. Alternatively, binding of the analyte to the reagent may activate the fluorophore to fluoresce. In some embodiments, the fluorophore is oxidised by an analyte or analyte reaction product in the sample. Oxidisation of the inactive fluorophore may activate the fluorophore to fluoresce.

In embodiments where the reagent comprises a fluorophore, the fluorophore may comprise rhodamine, for example Rhodamine 110. Advantageously, one or more peptides can be coupled to Rhodamine 110's amino groups, in order to generate a Rhodamine110/peptide conjugate. Various Rhodamine110/peptide conjugates are available commercially, for example from, Thermo Fisher Scientific. Upon cleavage of the peptides, the Rhodamine emits a detectable fluorescent signal. The cleaved Rhodamine 110 may be excited at a wavelength of at least 490 nm and no more than 510 nm. In some embodiments the cleaved Rhodamine 110 is excited at a wavelength of 505 nm. The cleaved Rhodamine 110 may emit light at a wavelength of at least 521 and no more than 530 nm. In some embodiments the cleaved Rhodamine 110 emits light at a wavelength of 525 nm. Other suitable fluorophores will be known to the skilled addressee. For example, other suitable fluorophores may include, but not be limited to FAM (fluorescein).

In the context of the present invention it will be understood that the term “peptide” refers to a compound comprising two or more amino acids linked in a chain.

In some embodiments the Rhodamine 110 is coupled to one or more peptides of fibrinogen to form a thrombin cleavable substrate. Rhodamine 110/fibrinogen peptide conjugates are available commercially, for example from Thermo Fisher Scientific as Rhodamine 110, bis-(p-Tosyl-L-Glycyl-L-Prolyl-L-Arginine Amide), catalogue number R22124. Upon contact with thrombin, the fibrinogen peptide arms of Rhodamine 110, bis-(p-Tosyl-L-Glycyl-L-Prolyl-L-Arginine Amide) are cleaved from the Rhodamine by thrombin. This advantageously converts the non-fluorescent bisamide Rhodamine into a fluorescently detectable form. The fibrinogen peptide arms are cleaved leaving initially a fluorescent monoamide and then fluorescent Rhodamine 110.

In embodiments where the reagent comprises a fluorophore, the fluorophore may comprise dihydrorhodamine 123. This is available commercially, for example from Thermo Fisher Scientific, amongst other commercial suppliers. Dihydrorhodamine 123 is an inactive fluorophore. Upon oxidisation, dihydrorhodamine 123 forms the fluorescent form cationic rhodamine 123. The cationic rhodamine 123 may be excited at a wavelength of at least 490 nm and no more than 510 nm. In some embodiments the cationic rhodamine 123 is excited at a wavelength of 488 nm. The cationic rhodamine 123 may emit light at a wavelength of at least 521 and no more than 540 nm. In some embodiments the cationic rhodamine 123 emits light at a wavelength of 530 nm.

In some embodiments the reagent comprises two fluorophores, for example CFP (cyan fluorescent protein) and YFP (yellow fluorescent protein), linked by a peptide. The peptide may comprise an enzyme cleavable peptide. Upon cleavage of the peptide, the first fluorophore becomes uncoupled from the second fluorophore. The first fluorophore can then transfer energy to the second fluorophore. Hence, if the peptide linker is intact, excitation at the excitation wavelength of the first fluorophore will result in the transfer of energy from the first fluorophore to the second fluorophore, resulting in the emission of a fluorescent signal from the second fluorophore in the process of FRET (Forster resonance energy transfer). If the peptide linker has been cleaved by the one or more components, FRET is abolished and emission is from the first fluorophore only.

The porous matrix may comprise the above described cleavable reagent at a concentration of at least 0.05 mM and no more than 1 mM, typically at a concentration of at least 0.1 mM and no more than 0.3 mM.

As above, it will be appreciated that concentration at a unit of mM relates to the concentration when provided in a liquid form, which is allowed to dry by evaporation or other means.

In some embodiments the porous matrix comprises the precursor of the analyte reaction product at a concentration of at least 0.05 mM and no more than 1 mM.

The analyte can be any desired analyte. In some embodiments the analyte is any desired analyte of whole blood. For example, the analyte may include one or more of proteins, peptides, antibodies, nucleic acid, microorganisms (such as bacteria, fungi and viruses), toxins, pharmaceuticals, enzymes, metabolites, lipids, peroxides, cellular moieties, antigens and the like.

In some embodiments the analyte comprises at least one enzyme. In a particular embodiment the analyte comprises at least thrombin.

In some embodiments the analyte comprises at least one lipid. In a particular embodiment the analyte comprises at least cholesterol.

In some embodiments the reagent/further reagent is in the form of a particle, comprising a binding, cleavage and/or interaction moiety and the moiety may be bound directly or indirectly, for example by non-covalent chemical bonding (e.g. biotin-avidin) to the surface of the particle. The particle may be a latex particle. In some embodiments the binding, cleavage and/or interaction moiety comprises Rhodamine110/peptide conjugate and the particle comprises a latex particle. In other embodiments the binding, cleavage and/or interaction moiety comprises dihydrorhodamine 123 and the particle comprises a latex particle.

Additional embodiments could also include physical adsorption, covalent chemical coupling, non-covalent chemical bonding (e.g. biotin-avidin) or any combination of these to the surface of a particle. Alternatively, the reagent/further reagent may be in free form, i.e. not bound directly or indirectly to a particle, for example free Rhodamine110/peptide conjugate or free dihydrorhodamine 123.

In some embodiments the reagent/further reagent comprises free Rhodamine110/peptide conjugate.

In some embodiments the reagent/further reagent comprises free dihydrorhodamine 123.

Transporting a sample along a microfluidic channel may comprise drawing the sample into the microfluidic channel by capillary action. In order that the sample may initially be introduced by way of capillary action, it is necessary for gas, which may be present in said microfluidic channel(s) to be displaced by the sample. This may be achieved by way of a valve or the like exiting from the microfluidic channel to outside of the cartridge.

Alternatively or additionally the sample may be actively drawn into the microfluidic channel by an active filling mechanism, such as described in WO 2018/002668. In such embodiments, the microfluidic channel may be connected to a gas chamber. Hence, in some embodiments a cartridge comprises a gas filled chamber(s) connected to a microfluidic channel(s). The gas chamber(s) and microfluidic channel(s) are thus understood to be in fluid communication with one another.

The sample may be actively drawn into and/or out of the detection zone by an active filling mechanism. In some embodiments, the sample may be actively drawn into and/or out of the section of the microfluidic channel comprising a porous matrix by an active filling mechanism.

Unless context dictates otherwise, the term “fluid communication” is understood to mean that a fluid, including a gas or a liquid, is able to be transported between the relevant parts.

Hence, the assay may further comprise compression and/or decompression of a/said gas filled chamber as described in WO 2018/002668 so as to control sample movement along said/each microfluidic channel.

The assay may further comprise a “stop flow” mechanism. This will be understood to define a mechanism wherein further flow of the sample is prevented once the sample reaches the detection zone and/or a stop feature, such as a hydrophobic printed feature downstream of the detection zone. Advantageously, this reduces the risk of the channel overflowing with excess sample and ensures that any analytes present are localised to the detection zone and the porous matrix, rather than flowing out of the detection zone and/or porous matrix. A particularly suitable mechanism is described in WO 2018/002668, to which the skilled reader is directed and the entire contents of which are hereby incorporated by way of reference. In summary, a piezoelectric bender(s) may be provided, which is/are designed to compress/decompress each/said gas filled chamber and thereby accurately control sample filling into the microfluidic channel and detection zone.

The sample may be introduced prior to the cartridge being inserted into the reader device or after the cartridge has been inserted into the reader. In some embodiments, the cartridge will be inserted into the reader device before the sample is applied and a force applied to the gas filled chamber in order to expel gas from said/each chamber. This may effectively prime the cartridge to be ready for sample application.

It is to be appreciated that compression and/or decompression of the/said gas filled chamber(s) may be carried out as a single or multiple steps, as described in WO 2018/002668. Compression of the gas filled chamber may be provided by a force application feature. The force application feature may be comprised within the reader device. Examples of suitable force application features and their presence in suitable reader devices may be found in WO 2018/002668 to which the skilled reader is directed and the entire contents of which are hereby incorporated. Other suitable force application features and/or reader devices can also be envisaged.

The gas within each chamber is typically air, although other gases or mixtures of gases may be introduced. For example, if any of the reagents which are deposited within each/said microfluidic channel(s) are liable to oxidation or otherwise possess a shorter lifespan when present in air, the cartridge and associated channels and chambers may be filled with an inert gas such as nitrogen, or the like. Generally reference will be made to the gas being air, but this should not be construed as limiting.

Advantageously, the careful control of the movement of gas into and out of said/each gas chamber is able to accurately control the rate of sample movement along each channel, in either direction. Moreover, the position of the sample within each channel can optionally be detected by the reader by means, such as electrodes, positioned along the microfluidic channels that are in contact with the reader and can feedback the position of any sample in each/said microfluidic channel thereby permitting the reader to very carefully determine the position and/or rate of sample movement through application of force/pressure to the gas/air filled chamber.

In one embodiment, the sample is initially introduced into the microfluidic channel(s) by capillary action to a first stop feature and thereafter further movement within the channel is controlled by an active force mechanism, as discussed above.

Thus, in a further aspect there is provided a kinetic assay method for use with a sample, the method comprising;
a) inserting a cartridge into a reader device, the cartridge comprising a microfluidic channel comprising:
a detection zone for receiving at least a portion of a sample provided to the microfluidic channel, the detection zone comprising an optically transmissible portion and a porous matrix comprising a reagent localised to an inner luminal surface of the optically transmissible portion, wherein the reagent is capable of reacting with an analyte in the sample or analyte reaction product thereof to form a reagent reaction product, and any reagent reaction product thereof being capable of being optically detected;
b) optionally expelling air from the microfluidic channel using force application means within the reader device;
c) introducing the sample to a first end of the microfluidic channel;
d) drawing the sample along the microfluidic channel by capillary and/or active force action to the detection zone;
e) permitting said analyte or analyte reaction product in the sample to react with the reagent;
f) taking at least one optical measurement of any generated reagent reaction product thereof, using an optical detector within the reader, the optical detector being extra luminal to the optically transmissible portion of the microfluidic channel; and
g) detecting any analyte or analyte reaction product based upon the at least one optical measurement of the reagent reaction product.

By localising the porous matrix comprising the reagent to the inner luminal surface of the optically transmissible portion, fluidic movement is accurately controlled without affecting the position or concentration of the porous matrix and any associated components, reagents and/or precursors.

The detection zone may be bordered by, for example hydrophobic features printed onto the portion, for example hydrophobic ink, carbon or silver. In this way the porous matrix is prevented from spreading outside the deposition zone by a border of features. In some embodiments the detection zone may be bordered by features printed onto a bottom layer, which function to limit further movement of the sample beyond the detection zone.

The printed features bordering the detection zone may also comprise electrodes. In some embodiments, once the sample reaches the printed features in step 1), the printed features trigger the taking of the optical measurement by the optical detection device and thus start the assay in step 2). Advantageously, this provides a start time to the assay which is independent of operator error. It also confirms that the sample has successfully filled the detection zone.

The sample may be a biological sample. The biological sample, for example whole blood, may be obtained from a subject prior to the assay. A subject may be predetermined for testing with the assay, or the subject may visit a healthcare provider, such as a doctor, nurse or other medical professional and the healthcare provider may identify the subject as requiring the assay to be conducted.

In a particular embodiment, the subject is taking or considering taking anticoagulant therapy. The subject may be showing symptoms or have a condition associated with coagulation, for example a stroke, deep vein thrombosis, a pulmonary embolism, or a myocardial infarction.

In a particular embodiment, the subject is taking or considering taking statins. In some embodiments, the subject has a family history of high cholesterol, smokes, is overweight, has diabetes, and/or has high blood pressure. The subject may have a family history of early cardiovascular disease.

The patient or healthcare provider may select a cartridge which is configured to carry out the assay and optionally, may insert this chosen cartridge into a reader. Alternatively, the biological sample may be sent by the healthcare provider or patient to an external provider to carry out the assay of the present invention.

The reader may configure itself appropriately in order to be able to run the assay and detect and/or determine the levels of the particular analyte present in the sample from the subject, based upon the optical measurement(s) obtained. The assay may be conducted on the sample, by the reader and cartridge working together and on completion of the assay, the reader will detect and/or determine the levels of the analyte which are present in the sample. The reader may then then provide the results of the assay to the subject and/or healthcare provider. The results may be provided by way of a visual result on a screen for example, or the results may be transmitted to another device such as a mobile phone/computer or the like. The results may also be presented as a print-out.

In some embodiments the result obtained may be converted to a standardised value, so as to be more informative. In such instances it may be necessary to calibrate the cartridge, reagents and/or reader. Thus, in some embodiments of the inventions, the assay method may comprise a calibration step or a calibration step is carried out prior an assay being conducted. The reader may be pre-programmed with appropriate calibration information such that results from a particular assay can be converted to a standardised value. Calibration steps are known in the art. Examples of calibration steps suitable for pT/INR assays can be found, for example, in Poller et al., Clinical Chemistry, 56:10, 1608-1617 (2010), to which the skilled reader is directed and the entire contents of which are incorporated herein by way of reference.

Typically, a calibration step for a PT/INR assay comprises the testing of a sample, for example a plasma sample with a certified INR in order to derive an ISI (international sensitivity index) value. The ISI value is indicative of the sensitivity of thromboplastin in the assay, and is used to calculate the standardized INR value. Suitable samples for deriving an ISI are commercially available, for example, lyophilised human plasma provided in an INR Correction Kit (Hart Biologicals, Hartlepool, UK).

Thus, in some embodiments the assay comprises a further step, the step comprising determining the levels of the analyte present in the sample. The level will be determined based upon the optical measurement(s) obtained.

When the analyte comprises thrombin, in some embodiments the further step comprises determining the levels of the thrombin present in the sample. The level will be determined based upon the optical measurement(s) obtained. The levels of thrombin may be detected or converted to a PT or INR value.

The results of the assay may be used to direct future diagnosis and/or treatment of the subject. Hence, it will be appreciated that the assay may include additional steps, directed to the selection or alteration of a particular therapy based upon the levels of the analyte determined from the assay. Desirably, the particular therapy may comprise anticoagulant or statin therapy. Suitable anticoagulant and statin therapy is known in the art.

As well as healthcare providers and/or patients, the user may be a law enforcement officer, or sport drug testing official, for example, where the subject is an individual being tested for inappropriate drug use.

As described above, in embodiments wherein the assay is a kinetic assay, a plurality of optical measurements is taken over a period of time of the analyte or the reaction product thereof. The plurality of optical measurements may comprise at least two, at least three, at least five, at least 10, at least 50, at least 100, at least 500, or at least 1000 optical measurements. In some embodiments, the plurality of optical measurements is taken continuously over a period of time.

Alternatively, an optical measurement may be taken over a period of time every 0.05 seconds, every 0.1 seconds, every 0.2 seconds, every 0.3 seconds, every 0.4 seconds, every 0.5 seconds, every 0.6 seconds, every 0.7 seconds, every 0.8 seconds, every 0.9 seconds or every 1 second. In some embodiments an optical measurement may be taken over a period of time every 0.1 seconds.

The period of time may be at least 10 seconds, at least 30 seconds, at least 1 minute, at least 1.5 minutes, at least 2 minutes, at least 3 minutes or at least 5 minutes. The period of time may be no more than 5 minutes, no more than 3 minutes, no more than 2 minutes, no more than 1.5 minutes or no more than 1 minute. In some embodiments the period of time is 2 minutes.

Since the plurality of optical measurements is taken over time, kinetic assays do not require an end point (i.e. a single measurement taken after a fixed incubation period). Advantageously, this provides a faster and more accurate assay than those previously known in the art.

The plurality of optical measurements may be taken until a threshold value is reached, after which no more optical measurements are taken.

The optical detector may be comprised within or connected to a reader device. In some embodiments the optical detector comprises a spectrometer or a fluorimeter. In some embodiments the optical detector comprises an LED (light emitting diode). Other suitable detectors will be known to the skilled person. For fluorescent detection the spectrometer or fluorimeter within the reader will detect direct or indirect fluorescence emitted from the analyte or the reaction product thereof within the detection zone. Thus, advantageously, only the optically transmissible portion of the cartridge which is designed to face towards the spectrometer or fluorimeter, or optical detection component, such as an optical fibre or fibres, connected to the spectrometer or fluorimeter, in the reader must be optically transmissible. In the case of fluorescence detection, the optically transmissible portion of the cartridge must be optically transmissible in the range encompassing an excitation wavelength and an emission (detection) wavelength. For example, the optically transmissible portion of the cartridge must be optically transmissible in the range 200-1200 nm.

As an alternative to a spectrometer or fluorimeter, optical detection may be carried out using a suitable camera, such as a CCD or CMOS camera, known in the art.

The microfluidic channel may have a depth of no more than 200 μm, no more than 150 μm, no more than 100 μm, or no more than 70 μm. The provision of such a depth advantageously ensures that the sample and the porous matrix are in close proximity. This depth also ensures that excitation wavelengths of light can reach the porous matrix, and that the emission wavelength can reach the optical detection device.

The optical measurement(s) may comprise a fluorescent measurement(s). Other suitable optical measurements will be known by the skilled person.

In some embodiments the assay may comprise a heating and/or cooling step. This may be separate to the present steps of the assay, or in addition to these steps. For example, prior to step 1) of the assay, the microfluidic channel may be heated. This heating may be maintained through one or more subsequent steps, or may be stopped once step 1) is initiated. Conversely, the microfluidic channel may be heated or cooled during any of the steps of the present assay. Heating or cooling may be provided by the associated reader device. It will be appreciated that the temperature of the assay is dependent upon the components to be detected/reaction occurring. The skilled person will therefore be aware of the optimum temperature for each assay/assay step.

The assay may comprise a heating step to heat and maintain the channel at a temperature of at least 20° C. and no more than 95° C. In some embodiments the assay may comprise a heating step to heat and maintain the channel at a temperature of at least 20° C. and no more than 65° C. The heating step may heat and maintain the channel at a temperature of at least 30° C. and no more than 40° C. In some embodiments the heating step heats and maintains the channel at a temperature of at least 35° C. and no more than 37° C.

In assays for determining a prothrombin time (PT) or international normalized ratio (INR) value, the assay may comprise a heating step to heat and maintain the channel at a temperature of approximately 30° C.-40° C., such as 36° C.

### DETAILED DESCRIPTION

The present invention will now be further described by way of example and with reference to the following figures which show:

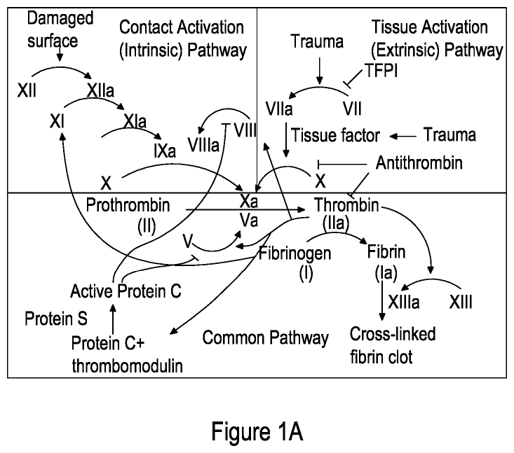

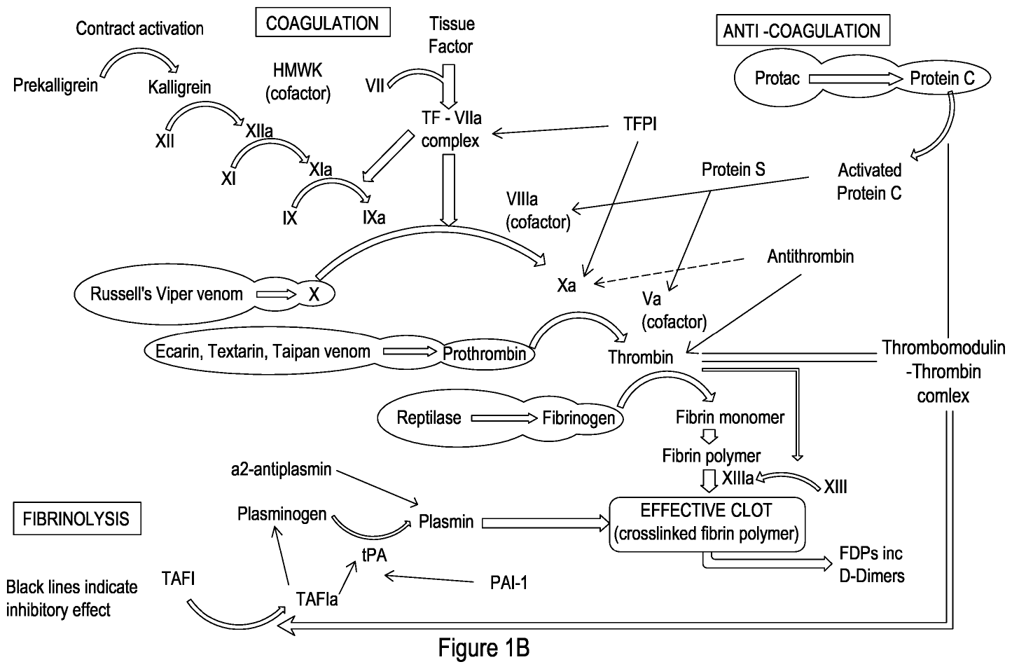

FIG. 1 (A and B) show a schematic representation of the coagulation pathway (or clotting cascade) and potential analyte targets of this pathway for measurement by a kinetic assay in accordance with the present invention;

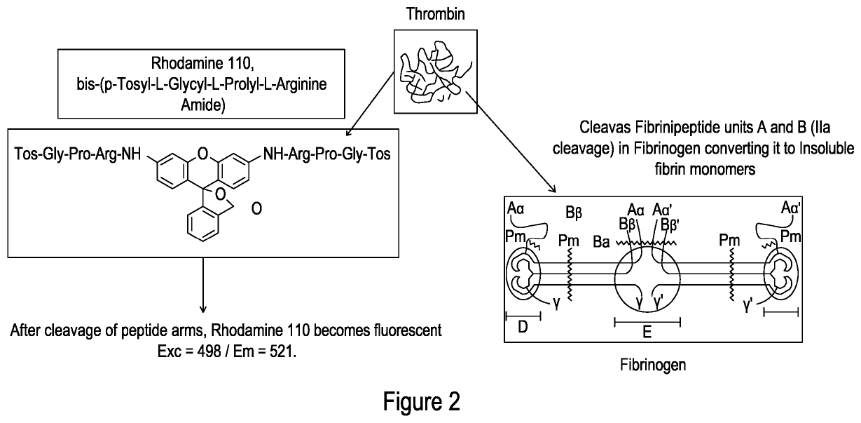

FIG. 2 shows a thrombin cleavable agent comprising Rhodamine 110, which may be used in an embodiment of the present invention;

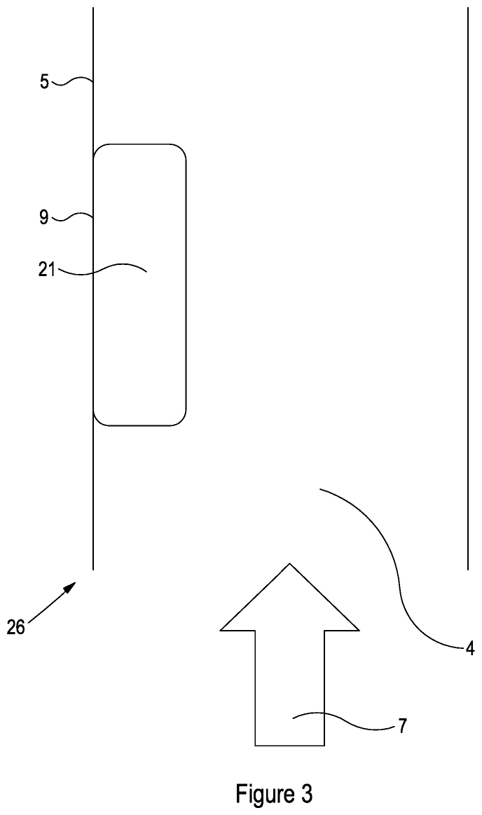

FIG. 3 is a schematic of an assay in accordance with an embodiment of the invention;

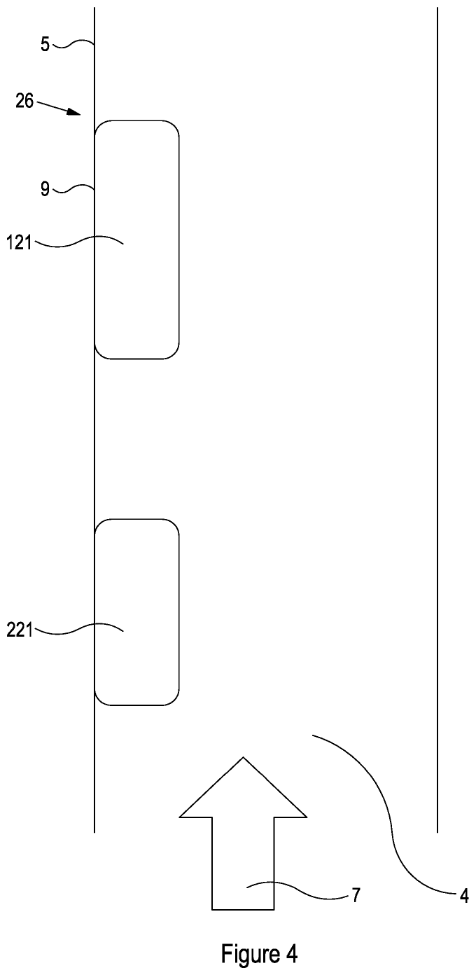

FIG. 4 is a schematic of another assay in accordance with an embodiment of the invention;

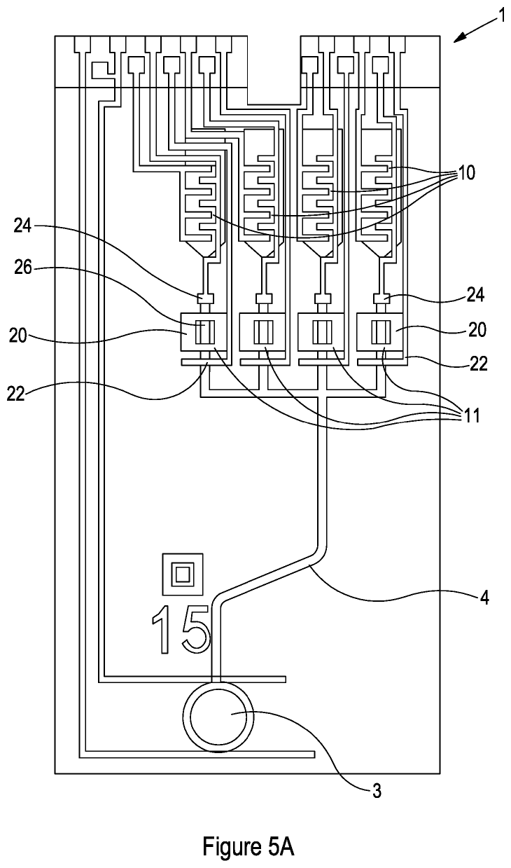

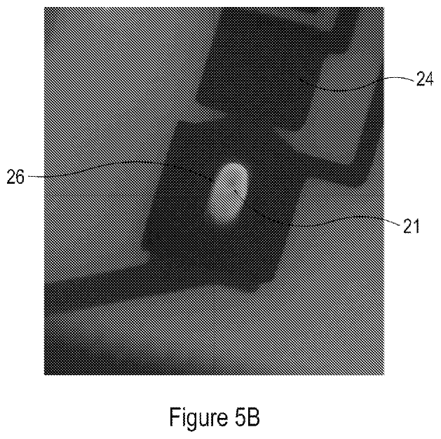

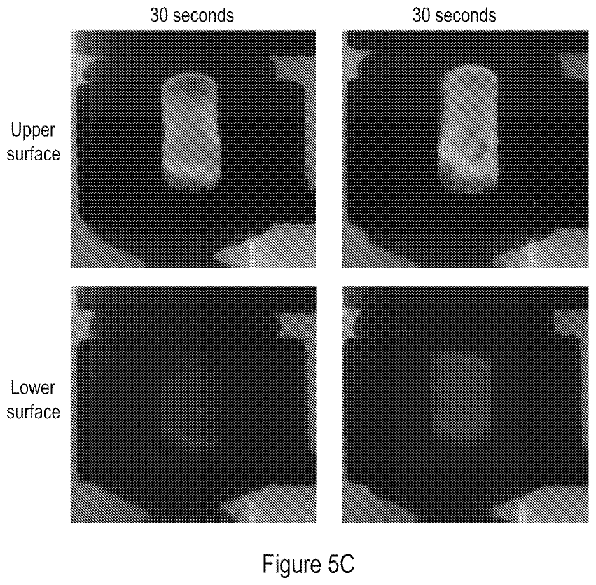

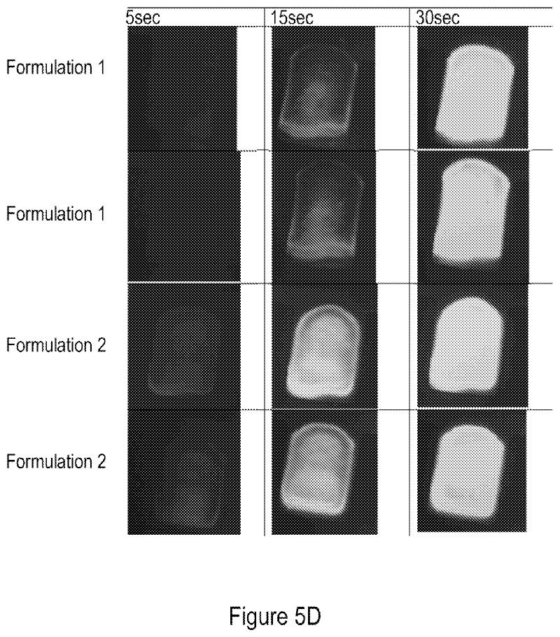

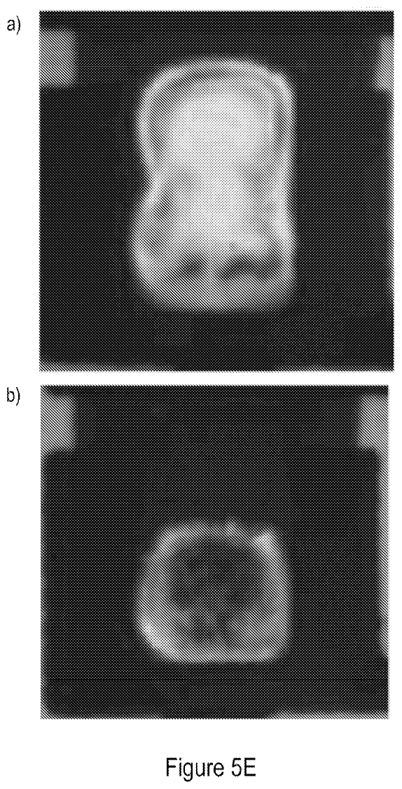

FIG. 5 (A) is a diagram of a microfluidic cartridge in accordance with an embodiment of the present invention. (B) is a photograph showing signal development at the optically transmissible portion during the assay. (C) shows the different signal intensity which can be obtained depending on where the porous matrix is positioned in relation to the optical detector. (D) shows signal development at different timepoints when using a porous matrix which was spread when deposited onto the luminal surface and (E) shows signal development when using a thick versus a thin porous matrix;

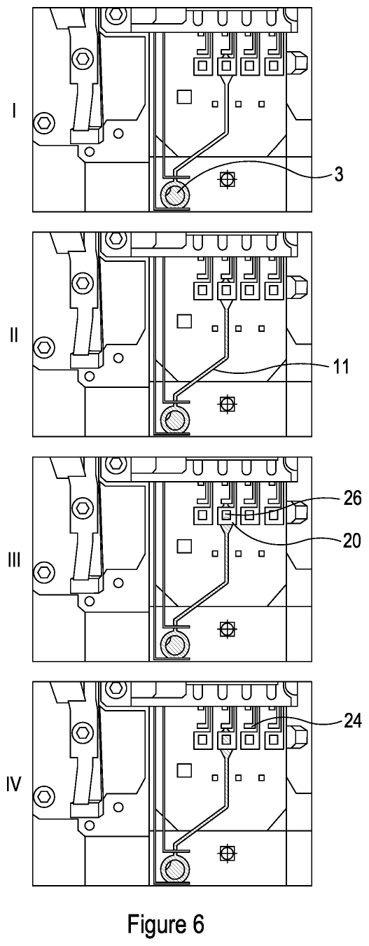

FIG. 6 shows blood entering and filling the portion of the cartridge shown in FIG. 5A;

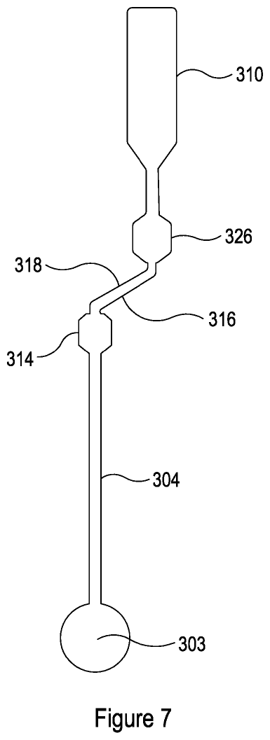

FIG. 7 is a diagram of microfluidic architecture in a cartridge in accordance with an embodiment of the invention;

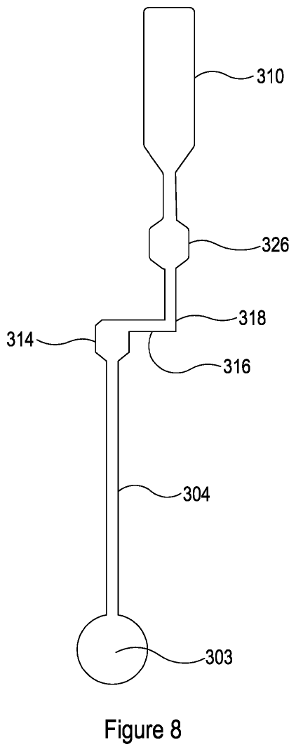

FIG. 8 is a diagram of microfluidic architecture in a cartridge in accordance with an embodiment of the invention;

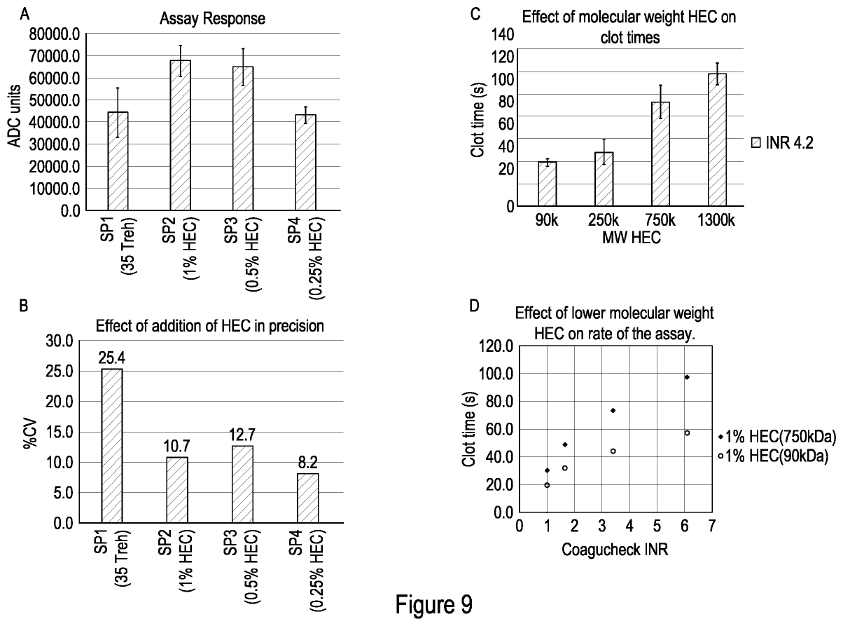

FIG. 9 (A and B) show the effect of HEC (hydroxyethylcellulose) as a component of the porous matrix on the optical measurement. (C and D) show the effect the molecular weight of HEC in the porous matrix has upon clot time;

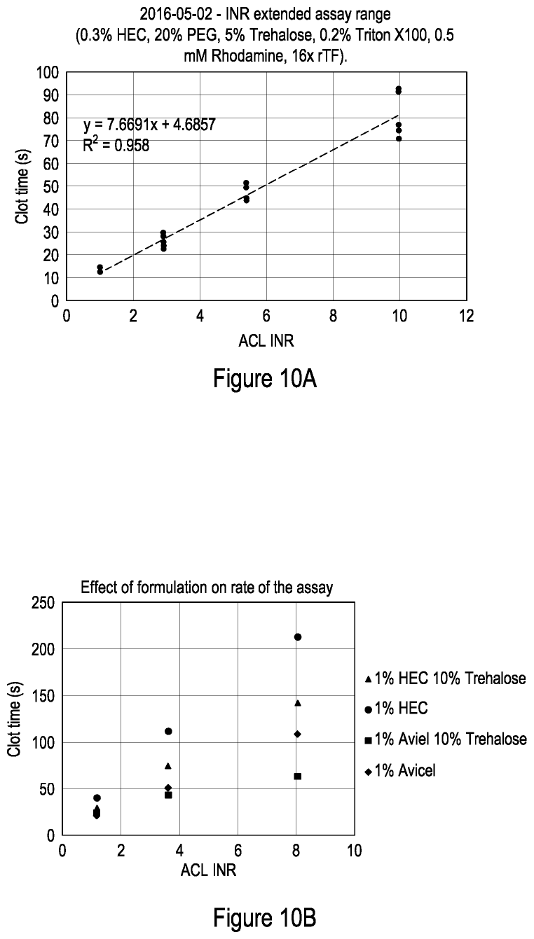

FIG. 10 shows the effect of other components of the porous matrix on optical measurement. (A) Clot time versus ACL INR on a sample of whole blood, the porous matrix having a concentration of 10% PEG (polyethylene glycol). (B) Effect of Avicel on assay rate;

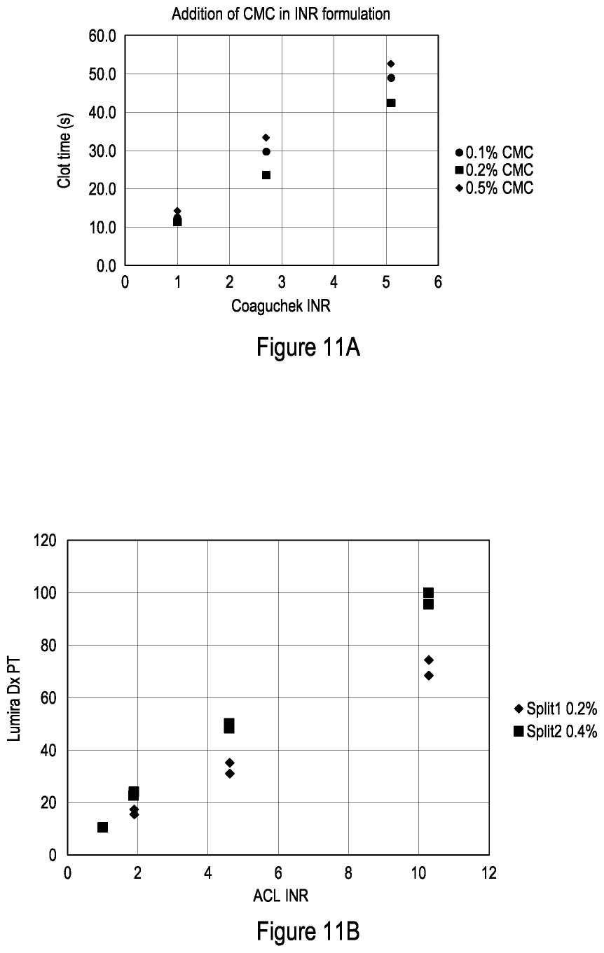

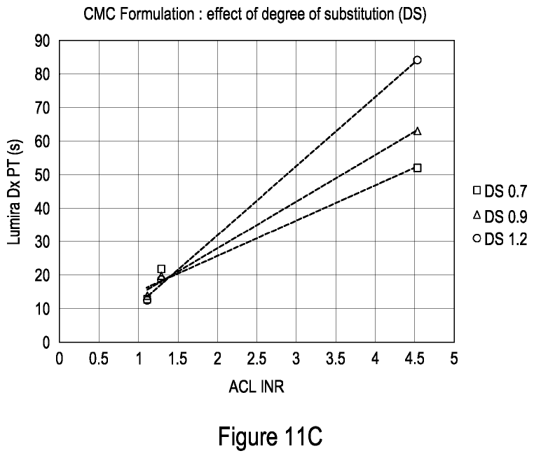

FIG. 11 shows the effect of CMC (sodium carboxymethylcellulose) in the porous matrix on the assay. (A and B) Effect of concentration of CMC in the porous matrix upon clot time. (C) Effect of new forms of CMC (DS (degree of substitution) CMC) upon the assay;

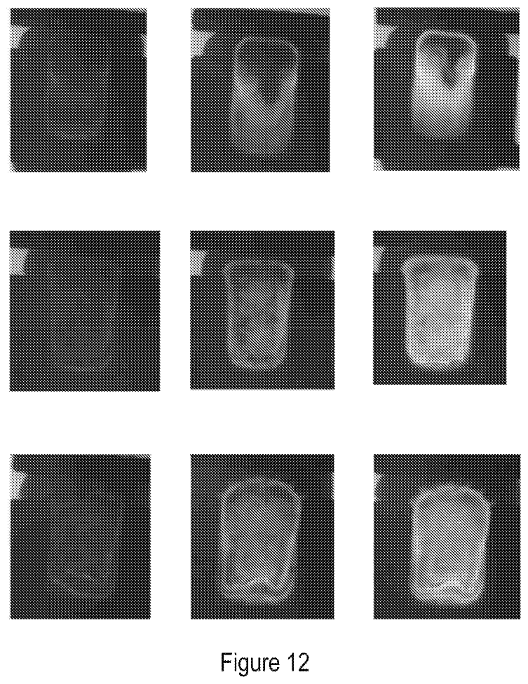

FIG. 12 shows images of signal development in the porous matrix for optical detection using different concentrations of CMC and PEG in the matrix;

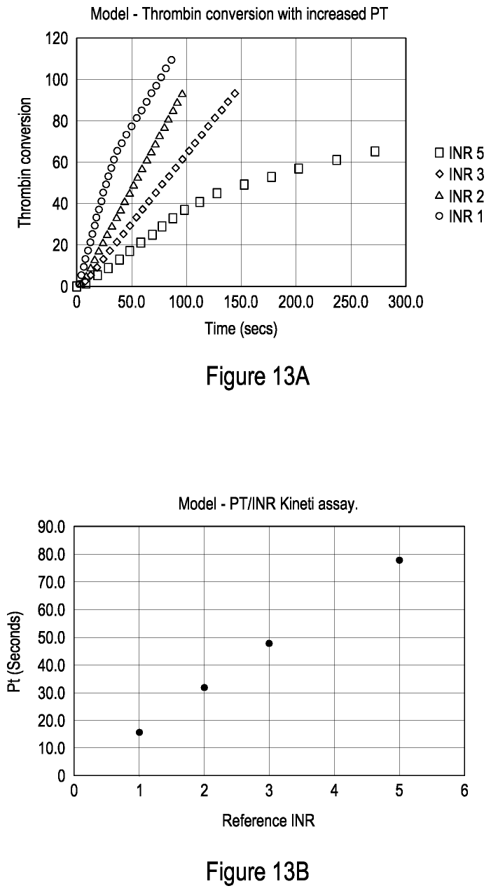

FIGS. 13 A and B show a modelled schematic of thrombin conversion as a measure of INR in accordance with an embodiment of the present invention;

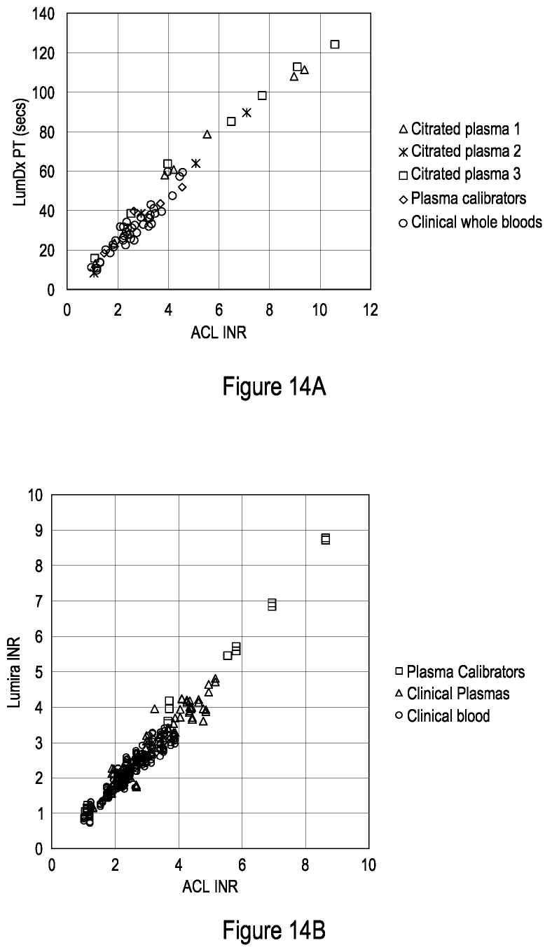

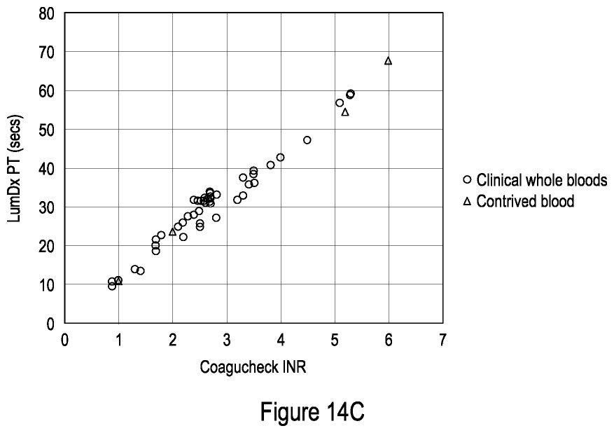

FIG. 14 shows assay results using different samples;

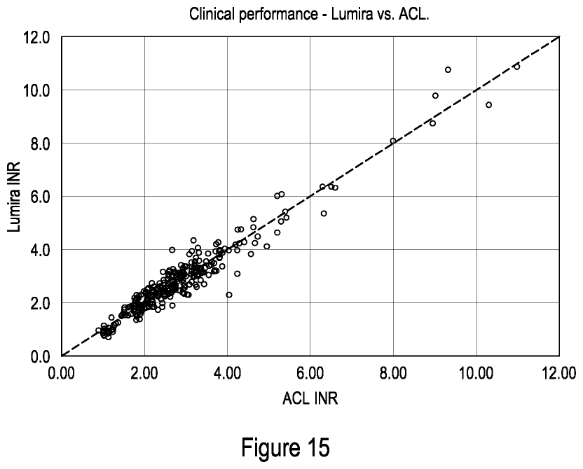

FIG. 15 shows the extended assay range of the present invention;

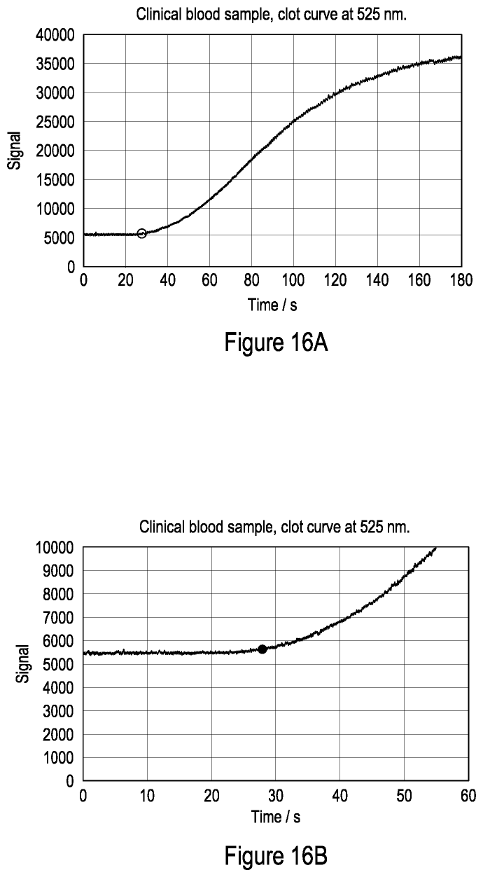

FIG. 16 shows a graph which demonstrates how clot time (and thus INR) may be calculated in an embodiment of the invention. PT is measured at clot onset, which is calculated as being 6×SD (standard deviation) over background;

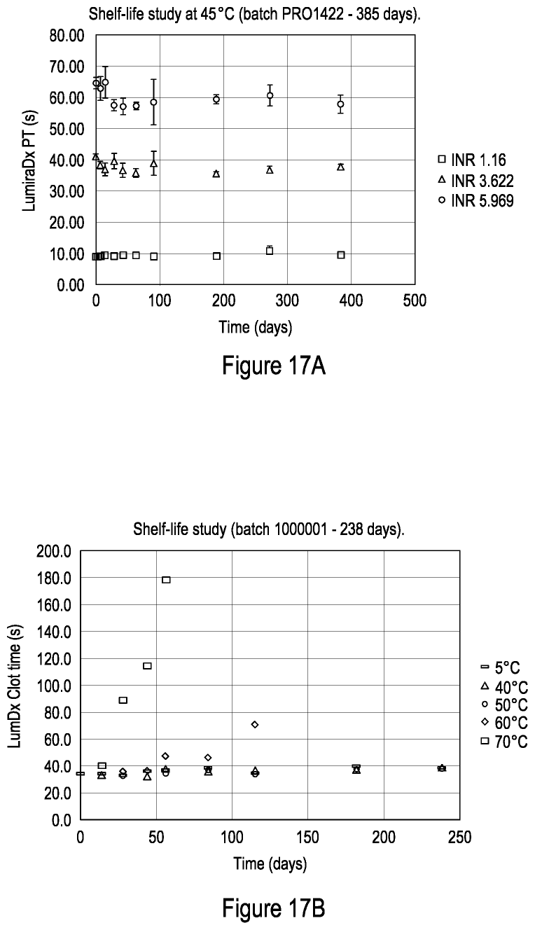

FIG. 17 shows the stability profile of the porous matrix after up to 13 weeks storage at 45° C. (A) and after up to 120 days at temperatures of between 5 and 70° C. (B);

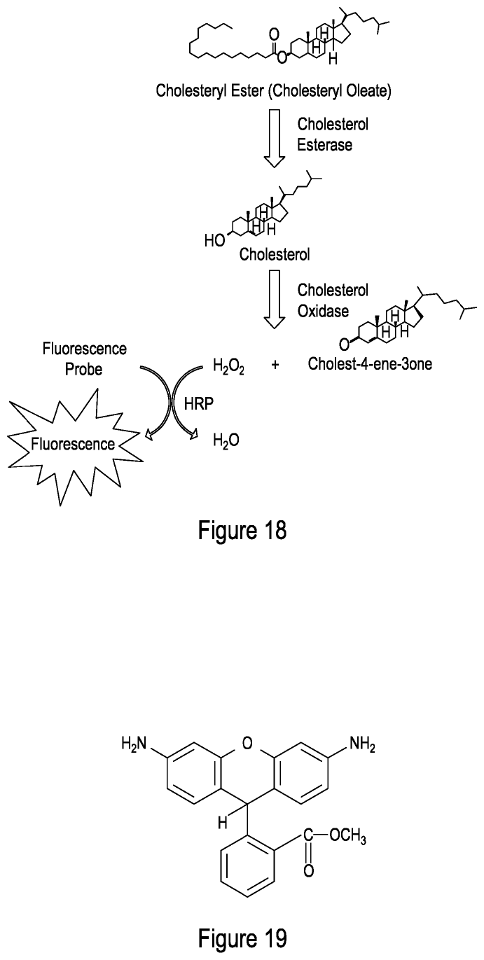

FIG. 18 is a schematic representation of the cholesterol pathway and potential reagents for use in detecting cholesterol in accordance with the present invention;

FIG. 19 is a diagram of dihydrorhodamine 123; and

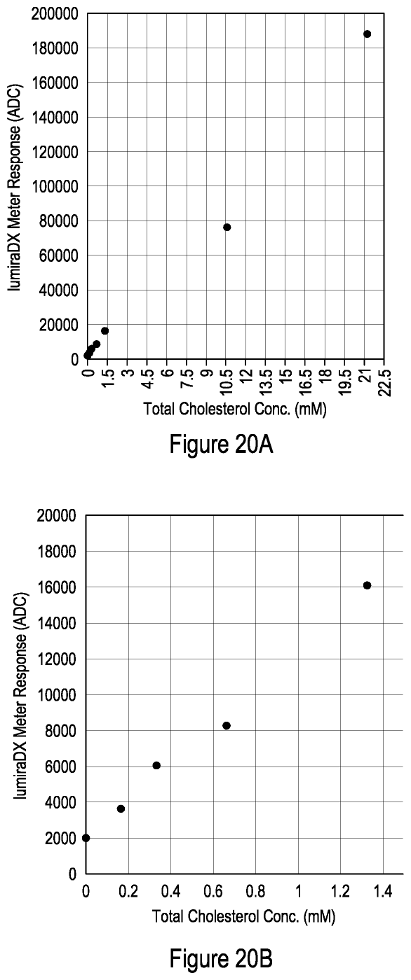

FIGS. 20 A and B show cholesterol assay results in accordance with an embodiment of the invention.

### EXAMPLE 1

FIG. 1 shows the analytes involved in the coagulation pathway, which is a series of enzyme assisted biochemical reactions that produce a blot clot. For many individuals, for example, patients taking anti-coagulant therapy such as warfarin or coumarin, the coagulation status must be regularly monitored. This enables the dose of therapy to be regularly adjusted to be within the therapeutic range while avoiding severe side effects such as increased bleeding.

As FIG. 1A shows, the cascade has two pathways, an intrinsic or contact activation pathway and an extrinsic or tissue factor pathway. The cascade is self-propagating once initiated, yet also exhibits self-regulation, since there are a number of inhibitors in the blood system which impede pro-coagulant steps and break down formed clots. The extrinsic and intrinsic pathways merge into the common pathway, at the centre of which is thrombin. This molecule has proteolytic activity, cleaving soluble fibrinogen and releasing insoluble fibrin which cross-links clots.

To monitor the coagulation status in known assays, a component is added to the patient sample which initiates clotting, for example, tissue factor. Tissue factor is a lipoprotein found only in tissue material and so is normally absent from the cardiovascular system. When a blood vessel is ruptured and no longer provides an effective barrier against tissue material, tissue factor enters the blood stream and the extrinsic cascade is initiated. One or more downstream analytes can then be measured. In the following embodiments, thrombin is the analyte which is detected by the assay of the present invention. However, because of the self-propagating nature of the cascade, numerous analytes of the pathway may be considered as potential biomarkers for coagulation status. As FIG. 1B shows, other factors, such as Russell's Viper Venom, Ecarin, Textarin, Taipan venom, Reptilase or Protac can be added to the patient sample to initiate clotting at different points of the pathway. There is accordingly the potential to monitor other components of the cascade, for example Factor X, Factor Xa, Factor II, Fibrin monomer or protein c. Thus, it is readily possible to envisage methods which are capable of detecting and/or determining the effect of other members of the extrinsic or intrinsic pathway, or other unrelated analytes.

Reagents Capable of Generating a Photometric Change

The present inventors found that a quenched reagent could be cleaved by thrombin, thus emitting fluorescence. FIG. 2 shows the thrombin cleavable agent, a bisamide derivative of Rhodamine 110 (Rhodamine 110, bis-(p-Tosyl-L-Glycyl-L-Prolyl-L-Arginine Amide, catalogue number R22124, Molecular Probes, Thermo Fisher Scientific). This reagent contains a fibrinogen peptide unit (peptide arm) covalently linked to each of Rhodamine 110's amino groups.

As shown in the Figure, in the cascade thrombin cleaves fibrinogen peptide units A and B, converting fibrinogen to insoluble fibrin monomers. The peptide arms attached to Rhodamine 110 replicate the fibrinogen unit peptides recognised naturally by thrombin. Thus, thrombin can cleave the peptide arms of the bisamide derivative of Rhodamine 110.

In an embodiment of the invention, the thrombin cleavable bisamide derivative of Rhodamine 110 is present within the porous matrix. The porous matrix is localised to an inner luminal surface of the optically transmissible portion of the microfluidic channel. It will, however, be appreciated that in other embodiments a porous matrix is not necessary. During the assay, the thrombin cleavable bisamide derivative of Rhodamine 110 comes into contact with a portion of the sample, for example blood. When thrombin is present in the sample of blood, thrombin cleaves the peptide arms of the bisamide derivative of Rhodamine 110. The cleavage of the peptide arms activates the bisamide derivative of Rhodamine 110 so that it emits an optical signal.

Describing the optical signal in more detail, as a result of cleavage of the peptide arms (Arg-Pro-Gly sequence), the non-fluorescent bisamide derivative Rhodamine 110 is converted first to a fluorescent monoamide and then to Rhodamine 110. Rhodamine 110 emits a fluorescent signal measurable on the wavelength of the optical detection device (excitation at 498-505 nm, emission at 521-525 nm). As the clotting cascade progresses downstream the conversion of prothrombin to thrombin increases and more Rhodamine 110 is released, therefore increasing the magnitude of the fluorescent signal. The conversion of prothrombin to thrombin is proportional to the fluorescent signal generated in a given time. The value for determining thrombin synthesis is known as prothrombin time (PT), or its derived measure, international normalized ratio (INR).

Hence, a high rate of fluorescent signal formation is indicative of a fast rate of prothrombin to thrombin conversion (low INR), while a low rate of fluorescent signal formation is indicative of a slow rate of prothrombin to thrombin conversion (high INR).

In a sample from a patient who is not taking anticoagulant therapy (non-therapeutic sample), the conversion of prothrombin to thrombin is very fast so a fluorescent signal is produced rapidly. In a therapeutic sample (a sample from a patient taking anticoagulant therapy), the cascade of reactions is slowed down, for example, in the case of Warfarin, due to inhibition of Vitamin K factors. Thus, PT is prolonged in patients on anti-coagulant therapy.

An example of such an assay is shown schematically in FIG. 3. In this Figure the porous matrix for generating an optical change/signal 21 is shown immobilised to the upper inner luminal surface 5 of a detection zone 26 of a microfluidic channel 4. A blood sample 7 enters detection zone 26, in which the porous matrix 21 is immobilised, permitting the blood sample 7 to come into contact with components including reagents within the porous matrix 21. Prothrombin within the blood sample 7 initially reacts with thromboplastin (TP) within the porous matrix 21, which initiates the clotting cascade and activation of clotting enzymes as shown. This results in the ultimate generation of thrombin which in turn is able to react with the bisamide derivative of Rhodamine 110 (R110) as discussed above, cleaving the peptide arm and generating an optically detectable signal within the porous matrix 21. The optical signal is detected using an optical detector present within an associated reader device, through an optically transmissible portion 9 of the detection zone 26. In this embodiment, the upper inner luminal surface 5 and the optically transmissible portion 9 are formed of the same optically transmissible material; however, in other embodiments it will be appreciated that the optically transmissible portion 9 may be formed of a different material to the rest of the upper inner luminal surface 5.

In the above example all the necessary components are present within a single porous matrix. FIG. 4 shows an alternative embodiment where some of the reaction components are separated into two porous matrices (121, 221). In this example, the first porous matrix 221 comprises thromboplastin (TP) for use in initiating the clotting cascade. The blood sample 7 initially comes into contact with the first porous matrix 221 in order to permit prothrombin within the blood sample 7 to come into contact with the thromboplastin within the first porous matrix 221 and the clotting cascade initiated. Thereafter the blood sample 7 is transported downstream to the second porous matrix 121 (the porous matrix for generating an optical change/signal) which comprises the reagent bisamide derivative of R110. Thrombin within the blood sample 7 cleaves the bisamide derivative of R110, thereby generating the reagent reaction product and an optically detectable signal, which in this embodiment, can be detected through the optically transmissible portion 9 of the detection zone 26 of the microfluidic channel 4.

It will be appreciated that although the embodiment of FIG. 4 shows only the second porous matrix 121 in the detection zone 26, in other embodiments the first porous matrix 221 can also be in the detection zone 26.

Separating or de-coupling the components/reagents such as exemplified schematically in FIG. 4 may be of use when the various components/reagents may potentially interfere with one another, or where it may be desired to alter the physical and/or chemical nature of each porous matrix. For example, it is possible to alter the porosity, wettability, pH etc. for each matrix and hence tailor this to the specific reactive components within each porous matrix. As made clear previously, it is readily possible for some reagents simply to be outside the porous matrix for optical detection. Thus, for example, a reagent could simply be dried down directly on to a surface of the microfluidic channel, or coupled with a carrier, but need not be present within a further porous matrix. Tests have shown this de-coupled embodiment to produce very reproducible results.

The close proximity of the optically transmissible portion 9/matrix 21 and the optical detector minimises any effect components which may be present in the sample may have in interfering with and/or obscuring optical detection. Without wishing to be bound by theory, the inventors also believe that the porous matrix is capable of excluding material of a particular size, such as red blood cells. This may reduce interference from such material during optical detection.

The assay performance of the free thrombin cleavable bisamide derivative of Rhodamine 110 (R110) (i.e. by “free” it will be appreciated that the bisamide derivative of Rhodamine 110 is not attached to another particle) when localised to the inner luminal surface of the optically transmissible portion was compared to magnetic or latex particles functionalised with the thrombin cleavable bisamide derivative of Rhodamine 110. Performance (measured as INR values), was comparable between the free thrombin cleavable bisamide derivative of R110 and latex particles functionalised with the thrombin cleavable bisamide derivative of R110. The performance of magnetic particles functionalised with the thrombin cleavable bisamide derivative of R110 was reduced compared to the other two groups.

It was decided to use the free thrombin cleavable bisamide derivative of R110 in further experiments since this removed the requirement of a solid phase, therefore reducing the complexity and cost of the porous matrix.

For the purposes of brevity, R22124 (catalogue number) as used herein refers to the free thrombin cleavable bisamide derivative of Rhodamine 110.

The effect of the concentration of R22124 within the porous matrix on the performance of the assay was also assessed. Concentrations (i.e. as provided in a liquid form, which is then allowed to dry by evaporation or other means in the matrix) of 0.1 mM, 0.25 mM, 0.5 mM, 0.75 mM and 1 mM R22124 were tested on contrived plasma samples and the results compared. A similar performance (as measured by clot time) was achieved from 0.25 to 0.75 mM; concentrations at 0.1 and 1 mM led to the highest clot times. Results are shown in Table 1.

TABLE 1
Effect of the concentration of R22124 (shown as [Rhod])
within the porous matrix on the performance of the assay.

| ACL INR | [Rhod] mM | Mean clot time | SD |
|---|---|---|---|
| 2.008 | 0.1 | 90.8 | 7.4 |
| 2.008 | 0.25 | 47.1 | 5.8 |
| 2.008 | 0.5 | 44.4 | 6.5 |
| 2.008 | 0.75 | 54.2 | 1.6 |
| 2.008 | 1 | 66.4 | 11.0 |

Microfluidic Channel/Cartridge

A sample microfluidic cartridge for use in the present invention is shown in FIG. 5a. In FIG. 5a, the cartridge 1 comprises a sample input port 3 connected to a microfluidic channel 4. The channel 4 extends within the cartridge 1 and branches to form a plurality of microfluidic channels 11, each channel 11 being fluidly connected to an independent gas filled chamber 10. In other embodiments the cartridge may comprise one, two or three channels 4.

The cartridge is formed from three separate planar layers, a first, second and third layer, in this embodiment a top and a bottom layer with a middle layer disposed between the top and bottom layers, which are sandwiched together to define the microfluidic channel 4 and the gas filled chamber 10. The middle layer is in the form of an adhesive layer which adheres the top and bottom layers. In the present embodiment, the channels of the cartridge are disposed within the middle layer. Hence, the channel walls are formed by the middle layer, and the base of the channels is formed by the bottom layer.

The cartridge of the embodiment of FIG. 5a is to be considered as self-contained; in that prior to application of a sample it is substantially liquid free and is considered as dry. The only fluid prior to application of the liquid sample which is present in this cartridge is a gas, in this embodiment air.

In use, a fluid sample (in the present embodiment, a blood sample) fills the channel 4 and this can be detected by electrodes which are in electrical contact with corresponding electric contacts within the reader. Upon the reader detecting an appropriate signal that a sample has been loaded into the cartridge 1 the reader can start the assays.

Describing each channel 11 in more detail, there are printed features (20, 22, 24) which are designed to limit movement of any reagent/porous matrix which is positioned within each channel 11 during the manufacturing process. The printed features (20, 22, 24) also function to limit further movement of the sample once it reaches the region of the printed features. In the present embodiment, the printed features are formed of a carbon-based hydrophobic ink and function as electrodes. In other embodiments, the printed features are formed of silver and can also function as electrodes. It will be appreciated that in other embodiments, the number of printed features may differ. For example, the printed features may comprise a printed feature bordering the detection zone (i.e. printed feature 20). Conveniently, the porous matrix is initially deposited within the area defined by the printed features and mechanically spread within. This limits and defines the area where the porous matrix is located The printed features may have a different shape to the shape shown in FIG. 5A. Printed feature 20 may be a different shape to the feature shown in FIG. 5A. For example, printed feature 20 may be a u-shape, i.e., open at one end. The printed feature 20 defines an area of 2×1.5 mm.

In use, an increasing force is applied to the chambers 10 of the cartridge 1, expelling gas from the chambers 10. Upon application of a blood sample to the cartridge, the sample is then drawn into the cartridge by air returning to the gas chambers 10 following a release in pressure being applied to the chambers (as will be described later). Once the sample reaches the printed feature 24 downstream of the detection zone, an increase in pressure is applied to the gas chambers to prevent further downstream movement of the sample.

In other embodiments, initial sample flow into and along the microfluidic channel take place by capillary action alone, to a first stop feature. Thereafter and in order to pass the stop feature, further sample flow is effected by an active force, as described above and below.

Located within the boundary of the printed feature 20 is a detection zone 26 of each assay channel 11 into which has been deposited a porous matrix (not shown) comprising a reagent designed to react with a particular analyte or reaction product thereof which may be present in a sample to be assayed. The porous matrix is optically transmissible and is localised to the inner luminal surface of an optically transmissible portion of the top layer. Multiple assays may be carried out on one microfluidic cartridge; the number of assays depends upon the number of separate channels and detection zones. In the present Figure four different channels 11 and detection zones 26 are shown.

Located further downstream to the detection zones 26 and printed features (20, 22, 24) are the gas filled chambers 10, which are designed to collocate with a force application feature present within a reader device (as will be described later) of the present invention, so that the force application feature is capable of applying a force to the gas filled chambers 10 so as to cause gas within the chambers 10 to be expelled from the chambers 10 and into the assay channels 11. A decrease in the force applied to the chambers 10 causes air to be drawn back into the chambers 10 from the assay channels 11.

Once the blood sample reaches the printed feature 24 which is located downstream of the detection zone 26, further movement of the blood sample is restricted. In addition, the printed feature 24 triggers the taking of at least one optical measurement by an optical detection device and thus the start of the assay.

Various reagents may be suitable for localisation within the porous matrix of the detection zone 26. For example localised in the porous matrix of the detection zones 26 may be free fluorogenic particles or fluorescently labelled latex particles functionalised with a further antibody designed to specifically bind a different epitope of analyte to be detected. In the present embodiment R22124, as shown in FIG. 2, is a reagent in the detection zone 26.

FIG. 5B shows a detection zone 26 once the assay of the present invention has started and detection of signal is ongoing. As shown in this Figure, a portion of a blood sample has reached the printed feature 24 which is located downstream of the detection zone 26, and so further movement of the blood sample is restricted. In this way, the portion of the blood sample is primarily localised to the detection zone 26 thus ensuring optimal detection. Optical measurements are taken using the LED (not shown), which is positioned above the detection zone 26. The matrix 21 remains localised to the inner luminal surface of the optically transmissible portion following the blood sample filling the detection zone 26. During the assay, no or very few red blood cells enter the matrix, as shown by the primarily clear matrix 21 in FIG. 5B. There is thus minimal interference with the optical detection of signal.

Although the above embodiment is shown with the porous matrix being immobilised on the upper intraluminal surface of the microfluidic channel and the optical detection taking place from above, it is possible for the porous matrix to be immobilised to the lower intraluminal surface of the microfluidic channel and detection taking place from below. In accordance with the invention detection should take place through the same surface to which the porous matrix is immobilised. Thus, if the porous matrix is immobilised to an upper intraluminal surface of the microfluidic channel, the optical detection system should not be arranged to detect any optical change through the lower intraluminal surface of the microfluidic channel and vice versa. That is, the bulk blood sample should not be positioned between the porous solid matrix and the optical detection system.

FIG. 5C shows the difference in signal intensity which can be obtained depending on where the porous matrix is positioned. The two top images show two repeats of the fluorescent intensity in the detection zone when a porous matrix according to an embodiment of the invention is retained on an inner luminal surface (in this case the upper surface) of the microfluidic channel, which is immediately adjacent to the optical detector present in the reader. The two bottom images show two repeats of the significant reduction in intensity displayed when the same porous matrix is positioned on the lower surface of the channel, when the optical detector is immediately adjacent to the upper surface. Hence the sample of blood within the channel is between the porous matrix and the optical detector. All images show the fluorescent intensity 30 seconds after a blood sample entered the detection zone. The same formulation was used for each porous matrix in each Figure. As can be seen, there is a distinct advantage in not having blood positioned between the porous matrix and the optical detector, in order to ensure efficient signal detection.

The porous matrix is initially spotted and contained within the boundary of printed feature 20 as shown in FIG. 5A. If simply spotted, the resulting dried spot will be generally round in nature and when dried will have an uneven thickness in cross section. That is the spot will be thinner at the edges of the spot, as compared to the center of the spot. The inventors have observed that by spreading the liquid spot, prior to it drying down, a more uniform thickness can be achieved, leading to a very good reproducible signal generation being achieved. FIG. 5D shows examples of different formulations of porous matrices in accordance with the present invention, in which each liquid spot has been spread and thinned prior to drying onto the surface. As can be seen good signal generation (from the reagent reaction product), over the three time points (5, 15 and 30 seconds after a blood sample entered the detection zone) across the entire spot is observed for each formulation. In this Figure, two porous matrices; a first porous matrix outside of the detection zone, and a second porous matrix for generating an optical change/signal in the detection zone, were utilised. In the top two rows, the porous matrix for generating an optical change/signal (the second porous matrix) comprised a formulation containing readiplastin, CMC, trehalose and rhodamine, amongst other components (formulation 1). In the bottom two rows of the Figure, the porous matrix for generating an optical change/signal (the second porous matrix) comprised a formulation which did not contain readiplastin (formulation 2). Both embodiments contained a further porous matrix (the first porous matrix) outside of the detection zone comprising trehalose and readiplastin. It will be appreciated that these formulations are exemplary and that other formulations can be used.

Signal development from a thin porous matrix as compared to a thicker porous matrix is shown in FIG. 5E. As is clear from this Figure, use of a thin layer (a) resulted in a fast and even reaction (see fluorescence), as compared to a thicker layer (b), where the reaction was slower and more patchy.

FIG. 6 shows a cartridge according to an embodiment of the present invention in use in the assay. A blood sample is contacted with and introduced into the cartridge 1 by way of the input port 3, as shown in step I of FIG. 6. The blood sample fills the channel 11 by the active fill mechanism, wherein following gas being expelled from the chamber 10 by pressure applied by the piezoelectric bender (not shown), the blood sample is drawn into the cartridge 1 and channel 11 by air returning to the gas chambers following a release in pressure being applied to the chambers 10 (step II and III of FIG. 6). The careful control of the reduction in force applied to the chambers 10 controls how far the blood sample is drawn into the detection zones 26, which are bounded by the printed features 20, and minimises sample variation effects on filling speed. This can also be controlled via electrode sensed feedback.

Further downstream of the detection zones 26 are the printed features 24, which are electrodes. The blood sample fills over the printed feature 20, and continues further downstream to meet the printed feature 24 (step IV of FIG. 6). Once the blood sample meets the printed feature 24, this printed feature 24 sends a signal to turn on the LED. At least one optical measurement is then taken by the LED (detector) in the reader. Further movement of the blood sample is restricted by the printed feature 24 and the stop flow mechanism. By waiting until the blood sample meets the printed feature 24 before starting to take optical measurements, this provides a start time 0 to the assay which is independent of operator error. It also confirms that the blood sample has successfully filled the detection zone 26.

FIGS. 7 and 8 show schematically some of the microfluidic architecture of two embodiments which employ an initial capillary fill, followed by an active fill. Each embodiment shows an input port 303 connected to a first portion of microfluidic channel 304. At the end of each first portion there is an enlarged region 314, where a haematocrit value of the blood sample can be obtained. Suitable methods for obtaining haematocrit values are known, for example, from US006064474A and Van Kempen E J and Zijlstra W. G et al. 1983 (Spectrophotometry of hemoglobin and hemoglobin derivatives. Advances in Clinical Chemistry, Vol 23, 199-257.) Extending downstream from the enlarged region 314 is a second portion of the microfluidic channel 316, which contains a small vent arm 318. This type of vent arm is described in more detail in WO2018/002668 to which the skilled reader is directed.

In use, once a sample of blood is introduced to the input port 303, the blood sample will fill the first portion 304, enlarged portion 314 and second portion 316 by capillary action, until encountering the vent arm 318, at which point capillary flow of the blood will stop. In order for further blood flow to occur, an active fill mechanism is utilised, as described above. Briefly, the active fill involves depression of the air/gas filled chamber 310. This causes the blood sample to flow past the vent arm 318 and into the detection zone 326 comprising one or more porous matrices (not shown), as previously described herein. The necessary reactions take place in the detection zone 326 and any optical change is detected using an optical detector as previously described.

The embodiment depicted in FIG. 8 is shown as having a longer second portion 316 before the detection zone 326. This readily accommodates the split or de-coupled method as described herein. For example, a first porous matrix, or one or more reagents may be deposited with the second portion 316 and further reagents or a second porous matrix comprising the reagents necessary for generating the optical change/signal, may be deposited in the detection zone 326. For example, the first porous matrix, or one or more reagents may be deposited in the section of the second portion 316 between the vent arm 318 and the detection zone 326. As previously discussed, in such a split matrices embodiment, the compositions of each matrix may be different and tailored to the requirements of the particular assay and reagents which are necessary. For example, for a PT/INR assay as discussed herein, the thromboplastin present in the first spot, may not need to be tightly bound within the spot, such that upon blood sample contact, it may be free to migrate from the porous matrix. However, the reagent which reacts to form the reagent reaction product (for example the bisamide derivative of Rhodamine 110), should be contained within a porous matrix, which substantially prevents or minimizes the reagent and the resulting reagent reaction product, from being washed out of the porous matrix. The inventors have observed that the use of CMC is particularly efficacious in this regard.

The above provides a description of one specific embodiment of the present invention, but the present invention is designed to be in the form of a platform assay which can easily be adapted. For example, the sample may be moved in the cartridge from capillary action alone, for example due to the addition of vents to the cartridge.

Moreover, multiple test formats may be assayed; cartridges with 2, 4 or 10 channel formats, for example, may be used.

Although the primary measurement technology is fluorescence the assay also incorporates electrochemical measurement and other components can easily be incorporated.

Various matrix formulations have been developed by the inventors for localisation to the optically transmissible portion to deliver accurate optical measurement of a sample. These are described in more detail below.

Other Components of the Matrix

Initial assessment of the matrix considered a simple thrombin-R110 method. In the assessment, R22124 was dissolved in a solution of ethanol (i.e. a wet assay). Lyophilised thrombin was solubilised in buffer and serial dilutions made. A volume of thrombin was reacted with the R22124 in ethanol in a cartridge, and after a period of incubation optical measurements were taken using a reader such as a reader described in WO2018/002668. A thrombin dose response curve was thus obtained. This confirmed that thrombin activity (and thus coagulation status) could be measured by a wet assay.

A dry matrix localised to the optically transmissible portion was then investigated. Free R110 was solubilised in DMSO or ethanol and deposited on the inner luminal surface of the optically transmissible portion of the microfluidic channel using trehalose as a carrier molecule for binding and to provide viscosity and stability. Trehalose is a non-reducing homodisaccharide in which two glucose units are linked together in an .-1,1-glycosidic linkage. Trehalose is classified as a kosmotrope or water-structure marker. It was found that this dry matrix returned a fluorescent signal when tested against lyophilised thrombin or plasma samples, although signal was low.

The effect of the concentration of trehalose in the matrix upon assay performance was assessed. The signals obtained from a sample with a known INR value of 3.5 using a matrix having a trehalose concentration of 20, 30, 40 or 50% (w/v) were observed. It was found that an increased trehalose concentration enhanced the signal. Later studies found that trehalose concentrations of 10 and 20% (w/v) were comparable.

Additional components of a dry matrix were then investigated. The effect of tissue factor upon assay performance was assessed. As previously noted, tissue factor is added to the patient sample to initiate clotting and thus to enable the determination of the coagulation status. Tissue factor was thus added to the formulation (which was then allowed to dry) at a concentration of between 1× and 8×.

To explain the 1×-8× nomenclature, RecombiPlastin 2G (Werfen) was used as the tissue factor. Product instructions recommend that the lyophilised RecombiPlastin 2G is dissolved in 20 mL of diluent. Werfen does not disclose the actual concentration of tissue factor in this product and so the initial concentration used in the formulation was not known.

Hence, to investigate the effect of tissue factor concentration, the lyophilised material was dissolved in lower volumes than recommended. Accordingly:

1×=20 mL diluent addition as recommended by the manufacturer;

2×=10 mL diluent;

4×=5 mL diluent;

8×=2.5 mL diluent.

16×=1.25 mL diluent

It was calculated that the concentration of tissue factor in the 16× concentration was 2189 pm.

Assessing a sample with a known INR of 7, it was observed that increased tissue factor in the matrix increased the rate of fluorescence generation of the assay.

In later studies the inventors switched from Recombiplastin 2G to Readiplastin. Clinical performance of Readiplastin was comparable to Recombiplastin 2G. Readiplastin is a concentrated liquid form of Recombiplastin 2G in which calcium has been removed

The effect of clotting factors upon assay performance was then assessed by measuring end-point fluorescence. Factor V/Va is a cofactor which forms the prothrombinase complex with Factor Xa and calcium. This complex cleaves prothrombin converting it to the active form, thrombin. Factor V is modulated by thrombin being directly activated by thrombin on a positive feedback and indirectly inactivated by thrombin through the thrombomodulin-protein C complex (with the cofactor protein S) pathway.

Factor Va was added to the sample at concentrations between 0 and 100 units/ml, and the effect upon the signal obtained from clinical plasma samples with different known INR values (5.0, 2.8 and 3.8) was observed. Adding factor Va primarily increased the signal observed. However, it slightly decreased the signal (fluorescence as measured at an end point) obtained in some samples at the higher concentrations (50 or 100 units/ml).

Factor X/Xa is another clotting factor which is activated by factor VIIa/tissue factor complex. Factor Xa binds to Factor Va and calcium to form the prothrombinase complex and converts prothrombin to thrombin. Factor Xa was added to the sample at concentrations between 0 and 15 μg/ml, and the effect upon the signal obtained from clinical plasma samples with different known INR values (7.8, 2.5 and 3.8) was observed. Adding factor Xa increased the signal observed, with a slight reduction of signal at 15 μg/ml only.

Prothrombin is the inactive zymogen of thrombin. The rate of conversion to thrombin is what assays measure to monitor clot time. Prothrombin was added to the sample at concentrations between 0 and 0.2 mg/mL, and the effect upon the signal obtained from clinical plasma samples with different known INR values (2.1, 3.9, and 6.5) was observed. An increasing concentration of prothrombin increases the signal observed.

Calcium, another clotting factor, was also investigated. Ca²⁺ was added to the sample at a concentration of between 0 and 25 mM, and the effect upon the signal obtained from plain whole blood samples was observed. Similarly to many other clotting factors, calcium had an initial enhancing effect upon signal followed by an inhibitory effect when concentration was too high (above 12 mM).

Other clotting factors investigated were Factors Vila, IXa and XIa.

One or more of these clotting factors could optionally be used in a quality control channel in a cartridge of the assay.

Studies were undertaken to assess the effect of different carrier molecules in the matrix on assay rate. Without wishing to be bound by theory, the inventors believe that the carrier molecule acts as a scaffold to hold R22124. The inventors also believe that the carrier molecule acts as a selective filter, allowing thrombin (or other analytes) to enter the formulation yet preventing red blood cells, or other particulate material/components from entering the matrix. Therefore, the interaction of R22124, thrombin and optionally tissue factor is favoured.

The effect of incorporating the polymer carrier molecule Hydroxyethylcellulose (HEC) into the matrix was investigated. HEC is a non-ionic water-soluble polymer made by reacting ethylene oxide with alkali-cellulose. Solutions of HEC are pseudo-plastic or shear-thinning. HEC is easily dissolved in cold or hot water to give crystal-clear solutions of varying viscosities. Furthermore, low to medium molecular weight types are fully soluble in glycerol and have good solubility in hydro-alcoholic systems containing up to 60% ethanol. HEC is generally insoluble in organic solvents. HEC can be found as a polymer of different lengths and molecular weights. In the present examples, HEC had an average molecular weight of 720,000 g/mol, but as the skilled person will appreciate, there are other commercially available molecular weights, for example 90,000, 250,000 or 1,300,000 g/mol.

FIG. 9 shows the effect of adding HEC as a carrier molecule of the matrix. FIG. 9A shows the end-point signal generated from a blood sample when either 35% trehalose or a concentration of 1%, 0.5% or 0.25% HEC was included in the matrix. As FIG. 9A shows, replacing trehalose with HEC increases the signal (i.e. end point fluorescence signal) generated. FIG. 9B shows the improved signal when HEC replaces trehalose in the matrix. The same concentrations were used in B as in A. End-point signals were obtained for FIGS. 9A and 9B using a reader as described in WO2018/002668

The effect of the molecular weight of HEC upon clot time, when used in the matrix, was then investigated. FIG. 9C shows that for a sample with an INR value of 4.2, decreasing the molecular weight of the HEC in the matrix decreased the clot time. The effect of the molecular weight of HEC upon clot time was further investigated as shown in FIG. 9D. In this Figure, samples with known INRs of between 0 and 8 (as measured using a reference system, in this Figure the CoaguChek® XS system, Roche, see x axis) were used in an assay according to an embodiment of the invention, wherein the formulation of the matrix contained either 1% HEC with a molecular weight of 750 kDa, or 1% HEC with a molecular weight of 90 kDa. In accordance with FIG. 9C, decreasing the molecular weight reduced clot times.

The inventors observed that in the absence of trehalose, HEC was very viscous. To aid coverage of the optically transmissible portion, it was therefore decided to add HEC and trehalose to the matrix to provide stability and aid deposition of the free R110. When in combination with HEC, Trehalose was included at lower concentrations than those previously studied, for example, 10%.

Other carrier molecules were also assessed. The assay was tested upon a whole blood sample using a matrix containing 0.3% HEC, 5% trehalose and 20% PEG (polyethylene glycol) (as well as Triton X 100, free R110 and tissue factor); clot times generated from this assay are shown in FIG. 10A. As this Figure shows, using the matrix described above, the clot time as determined by a method according to the present invention and the INR are linearly related.

An INR of 1 as measured by a reference system (ACL elite—plasma based lab analyser, Werfen, UK.) was detected in a reader according to the present invention in 10-12 seconds, whereas an INR of 10 was detected at between 70 and 90 seconds. The reference INR values were provided by reference plasma samples and appropriately diluted reference plasma samples in accordance with the Hart Biologicals INR correction kit (Hartlepool, UK). This shows that high INRs can be measured in less than 100 seconds using a method according to the present invention.

Avicel was also studied as a potential carrier molecule for the matrix. Avicel RC-591 is a spray-dried blend of microcrystalline cellulose (MCC) and sodium carboxymethylcellulose (CMC), which acts as a water dispersible organic hydrocolloid. In the presence of water and mild shear, the Avicel RC-591 powder particles swell and are then peptized, forming a dispersion of cellulose microcrystals. These microcrystals create a stable lattice structure.

Due to the small size of the microcrystals (approximately 60% of the crystallites in dispersion are ⋅0.2 μm), there are a large number of microcrystals packed in each powder particle. The large number of small microcrystal particles helps to promote a slower and more uniform settling rate, suspension stability and the absence of hard packing. It also provides dispersion and stability.

The effect on the assay rate of replacing HEC and/or trehalose with Avicel was studied. Contrived blood samples with known INR values were tested in an assay wherein the carrier molecule in the matrix localised to the inner luminal surface of the optically transmissible portion consisted of HEC and trehalose, HEC only, Avicel and trehalose or Avicel alone. As can be observed in FIG. 10B, the use of Avicel in the matrix reduced the assay times. Hence, an INR of 8 is detected approximately 140 seconds faster in an Avicel and trehalose matrix as compared to a matrix containing HEC.

It was then decided to study the effect of the polymer sodium carboxymethylcellulose (CMC) as a carrier molecule of the matrix when localised to an inner luminal surface of the optically transmissible portion, since CMC is a component of Avicel RC-591. FIG. 11A shows the effect of different concentrations of CMC (0.1%, 0.2% or 0.5%) in the matrix upon the clot time of the assay (see y axis) when tested upon samples with identical known INR values (the INR values previously determined using a CoaguChek® XS reference system, see x axis).

Samples tested were reference plasma samples with known INRs and appropriately diluted reference plasma samples in accordance with the Hart Biologicals INR correction kit (Hartlepool, UK). In this Figure CMC was used instead of Avicel RC-591. Assay times were found to be comparable to those achieved using Avicel RC-591; assay times were still within 10 to 60 seconds for INR values 1-5.

Further investigations were carried out regarding the effect of the concentration of CMC in the matrix. 0.4% CMC or 0.2% CMC was incorporated into the matrix, and assays carried out on samples with identical known INR values (as previously measured using a ACL INR reference system). A CMC concentration of 0.4% resulted in longer clot times (shown plotted as seconds against the y axis) than those observed from a CMC concentration of 0.2% (FIG. 11B). For both CMC concentrations the assay was able to measure a wide range of INRs (INRs ranging from 1 to 10.3 are shown in FIG. 11B).

The CMC used in FIGS. 11A and B was a blend of polymers with unknown weight and purity. Following a further search, some pure forms of CMC were identified with differing degrees of substitution. The degree of substitution (DS) of a polymer is the average number of substituent groups attached per base unit (in the case of condensation polymers) or per monomeric unit (in the case of addition polymers). Some of the commercial CMCs used have a DS of 0.7 (i.e. an average of 7 carboxymethyl groups per 10 anhydroglucose units). Assays were performed on samples with identical INR values using CMC at differing DS' (0.7 DS, 0.9DS or 1.2 DS) in the formulation. FIG. 11C shows the clot times from these assays (see y axis), as plotted against the known INR values (as previously determined using an ACL reference system). All three CMC forms provided similar assay rates, but it was observed that increasing the degree of substitution prolonged clot times.

Combinations of carrier molecules for the porous matrix for optical detection were further considered. The effect of differing concentrations of CMC and PEG, and the ratio of CMC to PEG in the porous matrix for optical detection was assessed. In particular, the solubility of the matrix and hence the distribution of the reagent for detection was considered.

Formulations tested comprised PEG at a concentration by weight of between 0.83% and 3.33% and CMC at a concentration by weight of between 0.08% and 0.12% The formulations were used in a porous matrix for optical detection. The porous matrix, in addition to other components, comprised the fluorescent reagent R22124. Samples of blood having a contrived INR of 2.7 were used to test the different ratios of CMC to PEG in the matrix.

It was observed that increased fluorescence, and distribution of fluorescence occurred when the ratio of PEG to CMC was higher, i.e. when the porous matrix comprised a low concentration of CMC and a higher concentration of PEG. These results (0.08% CMC and 0.83% PEG or 0.08% CMC and 1.67% PEG) are shown in bottom two rows of images in FIG. 12.

The images in FIG. 12 show fluorescence detected in the porous matrix in the detection zone 20 seconds, 30 seconds and 40 seconds (from left to right, consecutively) after the blood sample entered the detection zone. In this embodiment, only one porous matrix, a porous matrix for optical detection in the detection zone, was used.

The top row of images show the fluorescence observed when the porous matrix comprised 0.08% CMC and 0.83% PEG. The middle row of images show the fluorescence observed when the porous matrix comprised 0.08% CMC and 1.67% PEG. The bottom row of images show the fluorescence observed when the porous matrix comprised 0.08% CMC and 3.33% PEG.

Without wishing to be bound by theory, the inventors believe that PEG may assist in the wettability of the porous matrix, while CMC may assist in the binding nature of the matrix, i.e. in localising the matrix to the surface of the microfluidic channel. By optimising the ratio of the two components, this may achieve a matrix with excellent localisation to the surface with improved wettability to allow the even distribution of the analyte and resulting reaction product. This may help to improve fluorescent signal.

Another molecule which the inventors decided to study was heparin. Heparin is an anticoagulant which prevents the formation of blood clots. Two types of heparin are currently available: unfractionated heparin (UFH) and low molecular weight heparin (LMWH). Heparin binds to the enzyme inhibitor antithrombin III (AT), causing a conformational change which results in its activation through an increase in the flexibility of its reactive site loop. The activated AT then inactivates thrombin, factor Xa and other proteases. The rate of inactivation of these proteases by AT can increase by up to 1000-fold due to the binding of heparin.

A known inhibitor of heparin is hexadimethrine bromide (HMB or polybrene). It was decided to assess the effect of the heparin enoxaparin upon the clot time of samples with known INR values in the assay. Addition of the heparin enoxaparin at a concentration of 3 U to the matrix (when wet, which was then allowed to dry) increased clot times as compared to assays without enoxaparin (samples with known INR values were tested). For example, when testing an INR value of approximately 4.4, the clot time when the matrix included enoxaparin was approximately 70 seconds longer than the clot time when the matrix did not include enoxaparin. The addition of 0.25 mg/mL polybrene inhibited this effect, such that clot times obtained from assays where enoxaparin and polybrene were included in the matrix were comparable to clot times from control matrices.

The dry formulation of the matrix brings a number of important differentiating advantages. Firstly, the dry formulation is viscous and can withstand blood flow; as a result the matrix remains aligned with the optical detection device for optimal detection. Without being bound by theory, the present inventors believe that the viscous nature of the matrix acts as a polymer scaffold to hold the active components of the matrix.

The dry matrix is porous. The porous nature of the matrix allows the ingress of necessary analytes or analyte reaction products, in this embodiment thrombin, while preventing the entry of larger components in the sample, such as red blood cells. This leads to a fast and sensitive assay.

By using a dry matrix, the matrix can be localised to the inner luminal surface of the optically transmissible portion of the microfluidic channel; the matrix is thus in close proximity to the optical detection device, for example an LED. This prevents optical interference. Due to the close proximity of the matrix to the optical detection device, the assay has an improved sensitivity to known assays. Without wishing to be bound by theory, the present inventors believe that this allows a much faster assay than those previously known in the art; interference by other components in the sample is overcome since the assay can kinetically detect thrombin activity.

In addition, the matrix is very viscous. Hence, while porous, the matrix remains in position even when in contact with the sample, for example a liquid. This ensures that the reagent/reagent reaction product thereof is localised in close proximity to the sample, further facilitating a rapid and sensitive assay.

Real-Time Kinetic Measurement

The close proximity of the matrix, the sample and the optical detector means that the conversion of prothrombin to thrombin, as detected by fluorescent signal, can be detected kinetically.

In the present assay, the rate of conversion from prothrombin to thrombin is faster in samples with low INR values (e.g. a value of 1 INR: i.e. a higher level of coagulation) than for samples with high INR values (e.g. a value of 10 INR: i.e. a lower level of coagulation). Accordingly, the present assay does not require an end point (i.e. a single measurement is performed after a fixed incubation period—although this is envisaged within the scope of the present invention) and so the assay may instead be stopped after reaching a threshold value or a certain rate. This results in a fast and efficient assay.

Once the sample has successfully filled the detection zone, this provides a start time of 0, and the taking of a plurality of optical measurements by the reader is triggered. During the taking of the plurality of optical measurements, time is counted as clotting time. The increasing fluorescence due to the continuing conversion of prothrombin to thrombin can continue until a threshold value is reached. Once the threshold signal is reached, the taking of optical measurements stops.

FIG. 13A shows a schematic model based upon the assay of the present invention. This Figure demonstrates that high levels of thrombin conversion (i.e. from prothrombin to thrombin) equate to low INR values, while low levels of thrombin conversion relate to high INR values. FIG. 13B is a schematic to demonstrate the linear relationship between clot time (PT) and INR values; a low clot time indicates a high level of thrombin conversion and a low INR value, while a high clot time indicates a low level of thrombin conversion and a high INR value. The samples tested were reference plasma samples (and appropriately diluted reference plasma samples with known INR values as provided by the Hart Biologicals INR correction kit (Hartlepool, UK).

Effect of Sample on Optical Detection

Many known assays require the separation of plasma from a whole blood sample before detection can occur. This is costly and time consuming. FIG. 14 shows that the present assay can be used to test various different samples including citrated plasma, plasma calibrators (for example, as provided by the INR correction kit as described herein) and clinical whole blood. The Figure also compares values obtained using an assay in accordance with the present invention as compared to other known assays.

FIGS. 14A and B shows results when testing various different samples including citrated plasma, plasma calibrators and clinical whole bloods. The same samples were also tested on an ACL device as described above. For FIG. 14A, the y axis label “lumira PT (secs)” represents the uncalibrated PT in seconds obtained using a method according to the present invention. For FIG. 14B they axis shows calibrated INR values determined using a method according to the present invention. The x axis label “ACL INR” represents INR values calculated using the ACL elite reference system.

FIG. 14C shows the uncalibrated PT in seconds obtained from clinical whole blood and contrived blood samples from a method according to the present invention (y axis) as compared to the INR values obtained from the same samples using a CoaguChek® XS system.

As is apparent from this Figure, the assay is equally effective when testing blood or plasma samples, and citration does not affect the results. Advantageously, the assay maintains linearity across a broad range of INR values.

Increased Range of Assay

The fast rate of the assay enables the measurement of very high INR samples with very low thrombin activities. Accordingly, the assay in accordance with an embodiment of the invention can advantageously measure a very broad range of INR values from clinical samples, as shown in FIG. 15. As shown in this Figure, linearity is maintained even at high INR values.

Calculation of INR

FIG. 16A shows how clot time (in relation to thrombin levels) was calculated in an embodiment of the present assay. Time 0 represents the time point at which the sample fills the detection zone. As the sample fills the detection zone, the dry porous matrix is wetted and the clotting cascade is triggered. This triggers the conversion of pro-thrombin to thrombin, and the subsequent cleavage of R22124 in the matrix to generate a fluorescent signal, in this instance with emission at 525 nm. The pro-thrombin time (or INR) value was measured at clot onset (red dot), which was defined as being 6× standard deviations over the averaged baseline. FIG. 16B represents a magnified form of the section of 16A showing the clot onset.

An alternative INR method is based on determining a PT value in order to derive an INR result through an assay calibration scheme. The PT represents a time in seconds taken for a sample to respond to activation of the extrinsic clotting cascade which is initiated by the thromboplastin contained in the cartridge.

A sequence of calculations is applied to the fluorescent signal data which is captured by the Instrument as the cascade reaction progresses—this sequence is referred to as the PT Algorithm.

The PT algorithm may be based on the following parameters and steps:

Code Parameters

| | |
|---|---|
| start_wl = 515 | # lowest wavelength to include |
| end_wl = 530 | # highest wavelength to include |
| threshold1 = −300 | # threshold for determining blood filling |
| threshold2 = 1000 | # threshold for the initial estimate of clot time |
| threshold3 = 200 | # threshold for the final estimate of clot time |
| loCut = 0.35 | # low level of regression data cut (e.g. 0.35 is 35%) |
| hiCut = 0.65 | # high level of regression data cut (e.g. 0.65 is 65%) |

PT time = Tclot − T0

Ts Determination: Ts is the initial fall in signal indicating arrival of sample and is caused by sample starting to move into the measurement chamber.

T0 Determination: T0 indicates the time where the detection zone is almost full of sample. It represents the start of mixing of sample with the reagents in the chamber and is used as the start of reaction time to calculate PT time.

T100 Determination: T100 indicates the signal has risen far enough that the point of clot formation is sure to be contained within the collected data point set.

T17000 Determination: the instrument shall proceed to Clot time Extraction below if a datapoint is found that is >17,000 count above the T0 value.

T100+30 s Determination: the instrument shall proceed to Clot time Extraction below if more than 30 seconds has elapsed since the T100 was detected.

Clot Time Extraction:

The Instrument shall perform a linear regression on the datapoints between T35 (loCut) and T65 (hiCut) to determine a slope and intercept for baseline correction.

Note: T35 is located 35% of the time between T0 and T100. T65 is located 65% of the time between T0 and T100 the reader shall apply the offset and slope to all datapoints to form a set of baseline-corrected datapoints.

The reader shall find the first point after T50 in the baseline corrected data at which the count value rises above 200 units.

The reader shall record this time as Tclot.

The reader shall calculate a PT time by subtracting the time value recorded for T0 from the time value recorded for Tclot.

The reader shall apply a calibration given by the calibration table for INR in the Lot Calibration File to the measured PT Time to obtain the INR value.

Summary:

As an alternative to a SD method described above, this is a fixed threshold with a correction of the baseline to ensure it is always flat.

There are also features of the transient that can be detected (drop, slope) as well as error trapping mechanisms to ensure all the expected steps are occurring as expected and related to the strip/assay/meter workflow (e.g. sample arrival within a given time, no breaching of the sample, slope reaches a certain signal within a given time, time outs, etc.)

Stability of Formulation

FIG. 17A shows the stability profile of a dry porous matrix according to an embodiment of the present invention (comprising tissue factor, rhodamine 11—and trehalose, amongst other components) after up to 39 weeks storage at 45° C. Storage of the dry matrix for up to 39 weeks at this temperature did not affect the clot times calculated by the assay when using such matrices and samples with known INR values.

FIG. 17B shows the stability profile of this dry matrix after up to 12 days storage at temperatures between 5 and 70° C. Clot time remained consistent even after storage of up to 60° C. for 120 days.

In embodiments comprising a plurality of porous matrices, the formulation of each porous matrix may be the same, or may be different. For example, the porous matrix for generating an optical change/signal may have a more viscous formulation and/or be less soluble that the other porous matrix/matrices. An exemplary porous matrices arrangement may comprise a porous matrix for generating an optical change/signal (i.e. a second porous matrix), having a low solubility formulation and a first porous matrix positioned outside of the detection zone having a low solubility formulation.

An example formulation for the porous matrix for generating an optical change/signal may comprise 0.20% CMC, 5% Trehalose, 33.1% thromboplastin (in embodiments directed to the detection of thrombin) and 0.125 mM R22124. The second porous matrix may comprise 5% trehalose.

When a plurality of porous matrices is envisaged, in some embodiments the porous matrices may not comprise PEG.

Other formulations can be envisaged.

### EXAMPLE 2

It is envisaged that the present invention may be used for other assays, for example kinetic assays such as the determination of cholesterol levels in a sample, for example whole blood.

FIGS. 18 and 19 provide an illustrative example of the pathway and components which may be utilised to detect cholesterol. The cholesterol for detection may comprise free cholesterol and/or cholesteryl esters.

If the analyte for detection is cholesteryl ester, the ester can be reacted with the enzyme cholesterol esterase to result in the generation of free cholesterol.

When cholesterol is in the presence of the enzyme cholesterol oxidase, the following chemical reaction occurs:

cholesterol+O₂⇄cholest-4-en-3-one+H₂O₂

H₂O₂ generated from the above reaction can be converted to H₂O by the enzyme Horse Radish Peroxidase (HRP), the by-product of which can be used to oxidise a reagent resulting in an oxidised reagent reaction product and the emission of an optical signal. By oxidising the reagent, this can result in the generation of an optical signal. In the present Example, the reagent is a fluorescent reagent, which emits fluorescence upon oxidation. In one embodiment, this fluorescent reagent is dihydrorhodamine 123, as shown in FIG. 19. Dihydrorhodamine 123 is an uncharged and nonfluorescent reactive oxygen species (ROS) indicator that can be oxidized to cationic rhodamine 123 which exhibits green fluorescence. Dihydrorhodamine 123 is commercially available from various suppliers, including, but not limited to, Thermo Fisher Scientific, Sigma-Aldrich and Abcam.

Hence, the porous matrix/matrices of the present invention can comprise the above described components in order to detect levels of cholesterol and/or cholesteryl esters. For example, for the detection of cholesterol, the porous matrix/matrices could comprise cholesterol oxidase, HRP and dihydrorhodamine 123. For the detection of cholesteryl esters, the porous matrix/matrices could additionally comprise the enzyme cholesterol esterase.

As described in Example 1, the components may be in one porous matrix, or may be separated or decoupled so as to reduce interference, or where it may be desired to alter the physical and/or chemical nature of each porous matrix. Suitable cartridge architecture for use in a cholesterol assay are shown in FIGS. 7 and 8, although it will be appreciated that other cartridge architecture can be envisaged.

An embodiment of a cholesterol assay is described below and in relation to FIG. 7.

A sample, for example blood is contacted with and introduced into the cartridge by way of the input port 303, which in this embodiment is circular (but it will be appreciated that other shapes can be envisaged).

Once a sample of blood is introduced to the input port 303, the blood sample fills the first portion 304, enlarged portion 314 and second portion 316 by capillary action, until encountering the vent arm 318, at which point capillary flow of the blood will stop. Further downstream movement of the sample of blood is controlled by an active fill mechanism, in which depression of the air/gas filled chamber 310 causes the blood sample to flow past the vent arm 318 and into the detection zone 326 comprising a porous matrix for generating an optical change/signal (not shown). In this embodiment, the porous matrix for generating an optical change/signal is immobilised to the upper luminal surface in the detection zone 326. It will be appreciated that in other embodiments, the porous matrix may be immobilised to the lower luminal surface.

The detection zone may be enclosed by a printed feature, which in this embodiment is used to detect the flow of the sample in order to determine a start time to the assay, as described above. In this embodiment, the printed feature is also used to control the active fill mechanism in order to stop, start, reduce or increase the active fill mechanism in order to change the flow rate and/or stop or start sample flow downstream.

Once the blood sample enters the detection zone 326, any cholesteryl esters present in the sample interact with the enzyme cholesterol esterase in the porous matrix to generate free cholesterol. The free cholesterol then interacts with the enzyme cholesterol oxidase in the porous matrix to generate cholest-4-en-3-one+H₂O₂. H₂O₂ is converted to H₂O by the enzyme Horse Radish Peroxidase (HRP) in the porous matrix, the by-product of which is then used to oxidise dihydrorhodamine 123, dihydrorhodamine 123 being present in the porous matrix. Oxidised dihydrorhodamine 123 emits an optically detectable signal, in this example a fluorescent signal. Any optical signal is then detected using an optical detector as previously described. Detection by the optical detector is initiated by the printed feature, which once it detects the sample in the detection zone 326 starts the assay.

In other embodiments, more than one porous matrix may be utilised. For instance, the channel may have a first portion (zone A), a second portion downstream of the first portion (zone B) and a third portion downstream of the second portion (zone C). A porous matrix may be positioned in each zone. A printed feature may separate each zone. Zone C may comprise the detection zone 326 in which is located the porous matrix for generating an optical change/signal. In zone A, the porous matrix comprises the reagent cholesterol esterase. The porous matrix in zone B comprises the reagent cholesterol oxidase and the porous matrix in zone C comprises the reagents dihydrorhodamine 123 and HRP. In such embodiments, the sample will fill the first portion of the channel 304 and fill downstream to zone A. In zone A, any cholesteryl esters present in the sample will interact with the enzyme cholesterol esterase in the porous matrix to generate free cholesterol. The free cholesterol will flow with the sample downstream to fill zone B, in which the free cholesterol will interact with the enzyme cholesterol oxidase to generate cholest-4-en-3-one+H₂O₂

These reaction products will mix with the sample which will then fill the downstream zone C. In the porous matrix of zone C H₂O₂ is converted to H₂O by the enzyme Horse Radish Peroxidase (HRP), the by-product of which is then used to oxidise dihydrorhodamine 123, which results in the generation of an optically detectable signal. Any optical signal can then be detected using an optical detector as previously described.

Alternatively, rather than having a zone A, B and C, the cartridge may have only a zone A and B and so only two porous matrices. In such embodiments the porous matrix in zone A may comprise cholesterol esterase and/or cholesterol oxidase. In embodiments comprising than one porous matrix, only one of the porous matrices may be deposited in the detection zone. However, in other embodiments more than one porous matrix may be deposited in the detection zone.

FIG. 20 shows the results of a cholesterol assay carried out in accordance with an embodiment of the invention. In this embodiment, a porous matrix was utilised, which was immobilised to an inner luminal surface of the detection zone. In this particular embodiment the porous matrix was immobilised to the lower inner luminal surface of the detection zone, but it will be appreciated that mobilisation to the upper inner luminal surface of the detection zone could instead be envisaged. The porous matrix comprised the components cholesterol esterase, cholesterol oxidase, HRP and dihydrorhodamine 123. The porous matrix contained trehalose as a carrier molecule.

A 10 μl sample containing a known concentration of cholesterol was added to the input port and the sample filled the channel to the detection zone. After 90 seconds of the sample being in the detection zone, an optical measurement was taken. Plots of the peak detected optical signal, in this embodiment a fluorescent signal (y axis), against total cholesterol concentration (x axis) are shown in FIG. 20A. FIG. 20B shows the lower cholesterol concentration values of FIG. 20B plotted as a smaller scale.

Exemplary Test Descriptions

Summary Test Sequence 1:
1. Cartridge Insertion into the reader.
2. Cartridge gas chamber compression by reader.
3. Sample application to the cartridge, filling by active fill (by partial chamber decompression) to the printed feature which is located downstream of the detection zone.
4. Wetting of the detection electrodes of the printed feature located downstream of the detection zone determines the test start timing and also initiates a stop-flow mechanism to prevent flow of the sample further downstream.
5. The sample wets the porous matrix localised to an inner luminal surface of an optically transmissible portion of the detection zone of the cartridge, the matrix containing a reagent capable of reacting with an analyte or analyte reaction product thereof of the sample.
6. The reagent reacts with the analyte/analyte reaction product in the sample to form a reagent reaction product which generates an optical signal.
7. A plurality of optical measurements is taken by an optical detector positioned extraluminally to the optically transmissible portion until a threshold signal is reached.
8. The gas chamber is further decompressed completely by a removal in force applied to the cartridge gas chamber. This removal of force completely forces all of the sample into the cartridge for subsequent safe disposal.

Summary Test Sequence 2:
1. Cartridge Insertion into the reader.
2. Cartridge gas chamber compression by reader.
3. Sample application to the cartridge, filling by active fill (by partial chamber decompression) to the printed feature which is located downstream of the detection zone.
4. Wetting of the detection electrodes of the printed feature located downstream of the detection zone determines the test start timing and also initiates a stop-flow mechanism to prevent flow of the sample further downstream.
5. The sample wets the porous matrix localised to an inner luminal surface of an optically transmissible portion of the detection zone of the cartridge, the matrix containing a reagent reacting with an analyte or analyte reaction product thereof of the sample.
6. The reagent reacts with the analyte/analyte reaction product thereof in the sample, to form a reagent reaction product which generates an optical signal.
7. An optical measurement is taken by an optical detector positioned extraluminally to the optically transmissible portion after a fixed period of time. Each strip batch and analyte channel is calibrated separately so the signal is transformed into component concentration.

Summary Test Sequence 3:
1. Cartridge Insertion into the reader.
2. Sample application to the cartridge, initial filling of the microfluidic channel by passive capillary fill and then active fill to the detection zone.
3. During filling of the microfluidic channel, the sample wets a first porous matrix localised to an inner luminal surface of the microfluidic channel, an analyte in the sample reacting with a reagent in the first porous matrix.
4. The analyte reaction product of the reagent and the analyte mixes with the sample which fills downstream of the first porous matrix to the detection zone.
5. The sample wets the second porous matrix for generating an optical change/signal localised to an inner luminal surface of an optically transmissible portion of the detection zone, the matrix containing a further reagent capable of reacting with the analyte reaction product.
6. The further reagent reacts with the analyte reaction product in the sample to form a reagent reaction product which generates an optical signal.
7. A plurality of optical measurements is taken by an optical detector positioned extraluminally to the optically transmissible portion until a threshold signal is reached.

Summary Test Sequence 4:
1. Cartridge Insertion into the reader.
2. Sample application to the cartridge, initial filling of the microfluidic channel by passive capillary fill and then active fill to the detection zone.
3. The sample wets the porous matrix for generating an optical change/signal localised to an inner luminal surface of an optically transmissible portion of the detection zone, the matrix containing a reagent capable of reacting with an analyte in the sample.
4. The reagent reacts with the analyte in the sample to form a reagent reaction product which generates an optical signal.
5. A plurality of optical measurements is taken by an optical detector positioned extraluminally to the optically transmissible portion until a threshold signal is reached.

Summary Test Thrombin Sequence:
1. Cartridge Insertion into the reader.
2. Cartridge gas chamber compression by force application feature of reader expels gas from the cartridge.
3. Blood sample application to the cartridge. The force applied by the force application feature to the gas chamber is reduced. This results in partial decompression of the gas chamber and the ingress of air back into the chamber. This pulls a portion of the blood sample along the channel by an active fill mechanism.
4. The portion of the blood sample continues to move downstream until it reaches the printed feature which is located downstream of the detection zone. This acts as a boundary to prevent further downstream flow of the sample and also initiates a stop-flow mechanism to prevent flow of the sample further downstream.
5. Wetting of the detection electrodes of the printed feature located downstream of the detection zone determines the test start timing.
6. The blood sample wets the porous matrix for generating an optical change/signal localised to an inner luminal surface of a optically transmissible portion of the detection zone, the matrix containing R110. The fibrinogen peptide arms of R110 are cleaved by thrombin present in the sample, generating a fluorescent monoamide which is excited by the light emitted by the optical detector, the fluorescent monoamide emitting a fluorescent signal at 525 nm.
7. A plurality of optical measurements is taken by the optical detector positioned extraluminally to the optically transmissible portion until a threshold signal is reached. The signal is transformed into component concentration.

Summary Test Thrombin Sequence 2:
1. Cartridge Insertion into the reader.
2. Blood sample application to the cartridge. The blood sample fills downstream into the microfluidic channel and detection zone due to a combination of passive capillary and active fill mechanisms.
3. The blood sample wets the porous matrix for generating an optical change/signal localised to an inner luminal surface of an optically transmissible portion of the detection zone, the matrix containing R110. The fibrinogen peptide arms of R110 are cleaved by thrombin present in the sample, generating a fluorescent monoamide which is excited by the light emitted by the optical detector, the fluorescent monoamide emitting a fluorescent signal at 525 nm.
4. A plurality of optical measurements is taken by the optical detector positioned extraluminally to the optically transmissible portion until a threshold signal is reached.
5. The gas chamber is further decompressed completely by a removal in force applied to the cartridge gas chamber. This removal of force completely forces the all of the blood sample into the cartridge for subsequent safe disposal.

Summary Test Cholesterol Sequence 1:
1. Cartridge Insertion into the reader
2. Cartridge gas chamber compression by force application feature of reader expels gas from the cartridge.
3. Blood sample application to the cartridge. The force applied by the force application feature to the gas chamber is reduced. This results in partial decompression of the gas chamber and the ingress of air back into the chamber. This pulls a portion of the blood sample along the channel by an active fill mechanism.
4. The portion of the blood sample continues to move downstream until it reaches the printed feature which is located downstream of the detection zone. This acts as a boundary to prevent further downstream flow of the sample and also initiates a stop-flow mechanism to prevent flow of the sample further downstream.
5. Wetting of the detection electrodes of the printed feature located downstream of the detection zone determines the test start timing.
6. The blood sample wets the porous matrix for generating an optical change/signal localised to an inner luminal surface of an optically transmissible portion of the detection zone of the cartridge, the formulation containing dihydrorhodamine-123. The dihydrorhodamine-123 is oxidised by hydrogen peroxide present in the sample, the hydrogen peroxide formed as a oxidisation product of cholesterol when catalysed by the enzyme cholesterol oxidase
7. The oxidised dihydrorhodamine-123 forms the reagent reaction product fluorescent form cationic rhodamine 123 which is excited by the light emitted by the optical detection device, the rhodamine-123 emitting a fluorescent signal at 530 nm.
8. A plurality of optical measurements is taken by an optical detector positioned extraluminally to the optically transmissible portion until a threshold signal is reached. The signal is transformed into analyte, in this embodiment cholesterol, concentration.

Summary Test Cholesterol Sequence 2:
1. Cartridge Insertion into the reader.
2. Sample application to the cartridge, initial filling of the microfluidic channel by passive capillary fill and then active fill to the detection zone.
3. During filling of the microfluidic channel, the sample wets a first porous matrix localised to an inner luminal surface of the microfluidic channel, cholesterol in the sample reacting with cholesterol oxidase in the second porous matrix.
4. The analyte reaction product of the cholesterol and the cholesterol oxidase mixes with the sample which fills downstream of the first porous matrix to the detection zone.
5. The sample wets the second porous matrix for generating an optical change/signal localised to an inner luminal surface of an optically transmissible portion of the detection zone, the matrix containing dihydrorhodamine 123, which is capable of reacting with the analyte reaction product. The second porous matrix also comprises HRP to facilitate this interaction.
6. Dihydrorhodamine 123 reacts with the analyte reaction product in the sample to form a reagent reaction product which generates an optical signal.
7. A plurality of optical measurements is taken by an optical detector positioned extraluminally to the optically transmissible portion until a threshold signal is reached.

1.-24. (canceled)

25. A kinetic assay method for use in detecting an analyte within a sample, the method comprising:
a) providing a sample to a detection zone of a microfluidic channel, the detection zone comprising an optically transmissible portion and a porous matrix comprising one or more reagent(s) localised to an inner luminal surface of the optically transmissible portion of the microfluidic channel, wherein the reagent(s) is/are capable of reacting with the analyte or a reaction product thereof to form a reagent reaction product, the reagent reaction product capable of being detected, using an optical detector which is extra luminal to the optically transmissible portion of the microfluidic channel;
b) taking at least one optical measurement of the reagent reaction product through the optically transmissible portion; and
detecting any analyte, based upon the at least one optical measurement of the reagent reaction product.

26. The assay method according to claim 25, wherein the optically transmissible portion of the channel is a top portion of the channel.

27. The assay method according to claim 25, wherein the microfluidic channel comprises at least one additional matrix and/or one or more assay reagents deposited outside the matrix.

28. The assay method according to claim 27, wherein said at least one additional matrix and/or one or more assay reagents is localized to a section of the microfluidic channel which is not the detection zone.

29. The assay method according to claim 25, wherein the matrix/matrices comprises at least one carrier molecule.

30. The assay method according to claim 29 wherein the carrier molecule is generally insoluble in the sample fluid.

31. The assay method according to claim 29 wherein the at least one carrier molecule comprises at least one polymer.

32. The assay method according to claim 31 wherein the at least one polymer comprises at least one disaccharide and/or polysaccharide.

33. The assay method according to claim 32 wherein the at least one disaccharide is selected from the group consisting of sucrose, lactose, maltose, trehalose, cellobiose and chitobiose and the at least one polysaccharide is selected from the group consisting of amylose, amylopectin, cellulose, cellulose derivative, chitin, callose, laminarin, chrysolaminarin, xylan, arabinoxylan, mannan, fucoidan and galactomannan.

34. The assay method according to claim 29 wherein the at least one carrier molecule comprises trehalose and at least one cellulose derivative, selected from the group consisting of carboxymethylcellulose (CMC), cellulose ethyl sulfonate (CES), hydroxyethylcellulose (HEC), hydroxypropyl methyl cellulose (HPMC), hydroxypropyl cellulose (HPC), microcrystalline cellulose (MCC), methylcellulose and salts thereof.

35. The assay method according to claim 25, wherein the sample is provided to the detection zone by an active fill mechanism.

36. The assay method according to claim 25, wherein the sample is a sample of blood or other bodily fluid.

37. The assay method according to claim 25 wherein the analyte is an enzyme, lipid, lipoprotein, cytokine, hormone or endotoxin.

38. The assay method according to claim 37 wherein the analyte is an enzyme.

39. The assay method according to claim 38 wherein the enzyme is thrombin or a protease.

40. The assay method according to claim 37 wherein the enzyme is thrombin and the method is used to determine a prothrombin time (PT) or international normalised ratio (INR) value.

41. The assay method according to claim 38 wherein the reagent comprises a thrombin cleavable substrate reagent, which is capable of being cleaved by thrombin to form a reagent reaction product which generates an optical signal.

42. The assay method according to claim 41 wherein the thrombin cleavable substrate reagent comprises a peptide sequence which is recognisable and cleavable by thrombin and an associated fluorescent molecule, the associated fluorescent molecule forming the reagent reaction product which is capable of detection following cleavage of the peptide sequence.

43. The assay method according to claim 37 wherein the analyte comprises a lipid or lipoprotein.

44. The assay method according to claim 43 wherein the analyte comprises cholesterol or a cholesteryl ester.

45. A microfluidic channel for use in a method according to claim 25, the microfluidic channel comprising:
a detection zone for receiving at least a portion of a sample provided to the microfluidic channel, the detection zone comprising an optically transmissible portion and a porous matrix comprising one or more reagent(s) localised to an inner luminal surface of the optically transmissible portion of the microfluidic channel, wherein the reagent(s) is/are capable of reacting with the analyte or an analyte reaction product thereof to form a reagent reaction product, the reagent reaction product being capable of being optically detected; and
optionally wherein the optically transmissible portion is a top portion of the channel.

46. A microfluidic cartridge for use in conducting an assay according to claim 25, the microfluidic cartridge comprising:
at least one microfluidic channel, wherein each/said microfluidic channel(s) comprises a detection zone, the detection zone comprising an optically transmissible portion and a porous matrix comprising one or more reagent(s) localized to an inner luminal surface of the optically transmissible portion, wherein the reagent(s) is/are capable of reacting with the analyte or an analyte reaction product thereof to form a reagent reaction product, the reagent reaction product being capable of being optically detected; and
optionally wherein the optically transmissible portion is a top portion of the channel.

47. An assay method for use with a sample, the method comprising;
inserting a cartridge into a reader device, the cartridge comprising a microfluidic channel comprising:
a detection zone for receiving at least a portion of a sample provided to the microfluidic channel, the detection zone comprising a optically transmissible portion and a porous matrix comprising one or more reagent(s) localized to an inner luminal surface of the optically transmissible portion, wherein the reagent(s) is/are is/are capable of reacting with the analyte or an analyte reaction product thereof to form a reagent reaction product; the reagent reaction product capable of being optically detected,
optionally expelling air from the microfluidic channel using force application means within the reader device;
introducing the sample to a first end of the microfluidic channel;
drawing the sample along the microfluidic channel to the detection zone using means within the reader device;
permitting said analyte or analyte reaction product in the sample to react with the reagent(s) to form the reagent reaction product;
taking at least one optical measurement of the reagent reaction product using an optical detection device within the reader, the optical detection device being extra luminal to the optically transmissible portion; and
detecting any analyte or analyte reaction product based upon the at least one optical measurement of the reagent reaction product.

48. The assay method according to claim 47, wherein the optically transmissible portion is a top portion of the channel.
